(19) United States
(12) Patent Application Publication
(10) Pub. No.: US 2006/0035884 A1
(43) Pub. Date: Feb. 16, 2006

(54) N-cyclic sulfonamido inhibitors of gamma secretase

(75) Inventors: Martin L. Neitzel, Pacifica, CA (US); Jennifer L. Marugg, San Jose, CA (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Elan Pharmaceuticals, Inc., South San Francisco, CA (US)

(21) Appl. No.: 11/133,738

(22) Filed: May 20, 2005

Related U.S. Application Data
(60) Provisional application No. 60/572,862, filed on May 20, 2004.

(51) Int. Cl. C07D 211/08; A61K 31/55

(52) U.S. Cl. 514/212.01; 514/317; 540/604; 546/192; 546/229

(57) ABSTRACT

The invention provides N-cyclic sulfonamido compounds for use in treating or preventing cognitive disorders, such as Alzheimer's Disease. 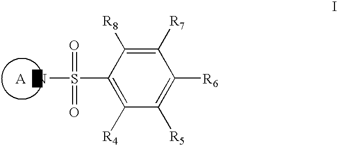
Compounds of particular interest are defined by Formula I, wherein R₄, R₅, R₆, R₇, and R₈, are as described in the specification. The invention also encompasses pharmaceutical compositions comprising compounds of Formula I as well as methods of treating cognitive disorders, including Alzheimer's disease using compounds of Formula I.

This invention claims priority from U.S. Provisional Application No. 60/572,862, which was filed on May 20, 2004, and is hereby incorporated by reference, it its entirety.

### BACKGROUND OF THE INVENTION

1. Field of the Invention

The invention relates to N-cyclic sulfonamido compounds which inhibit gamma secretase and β-amyloid peptide release and/or its synthesis. Therefore, the N-cyclic sulfonamido compounds are useful in the prevention of cognitive disorders in patients susceptible to cognitive disorders and/or in the treatment of patients with cognitive disorders in order to inhibit further deterioration in their condition.

2. State of the Art

Alzheimer's Disease (AD) is a degenerative brain disorder characterized clinically by progressive loss of memory, cognition, reasoning, judgment and emotional stability that gradually leads to profound mental deterioration and ultimately death. AD is a very common cause of progressive mental failure (dementia) in aged humans and is believed to represent the fourth most common medical cause of death in the United States. AD has been observed in races and ethnic groups worldwide and presents a major present and future public health problem. The disease is currently estimated to affect about two to three million individuals in the United States alone. AD is at present incurable. No treatment that effectively prevents AD or reverses its symptoms and course is currently known.

The brains of individuals with AD exhibit characteristic lesions termed senile (or amyloid) plaques, amyloid angiopathy (amyloid deposits in blood vessels) and neurofibrillary tangles. Large numbers of these lesions, particularly amyloid plaques and neurofibrillary tangles, are generally found in several areas of the human brain important for memory and cognitive function in patients with AD. Smaller numbers of these lesions in a more restrictive anatomical distribution are also found in the brains of most aged humans who do not have clinical AD. Amyloid plaques and amyloid angiopathy also characterize the brains of individuals with Trisomy 21 (Down's Syndrome) and Hereditary Cerebral Hemorrhage with Amyloidosis of the Dutch Type (HCHWA-D). At present, a definitive diagnosis of AD usually requires observing the aforementioned lesions in the brain tissue of patients who have died with the disease or, rarely, in small biopsied samples of brain tissue taken during an invasive neurosurgical procedure.

The principal chemical constituent of the amyloid plaques and vascular amyloid deposits (amyloid angiopathy) characteristic of AD and the other disorders mentioned above is an approximately 4.2 kilodalton (kD) protein of about 39-43 amino acids designated the β-amyloid peptide (βAP) or sometimes Aβ, AβP or β/A4. β-Amyloid peptide was first purified and a partial amino acid sequence was provided by Glenner et al., Biochem. Biophys. Res. Commun., 120:885-890 (1984) The isolation procedure and the sequence data for the first 28 amino acids are described in U.S. Pat. No. 4,666,829.

Molecular biological and protein chemical analyses have shown that the β-amyloid peptide is a small fragment of a much larger precursor protein termed the amyloid precursor protein (APP), that is normally produced by cells in many tissues of various animals, including humans. Knowledge of the structure of the gene encoding APP has demonstrated that β-amyloid peptide arises as a peptide fragment that is cleaved from APP by protease enzyme(s). Sequential processing of the precursor protein by the enzymes referred to generically as beta- and gamma-secretases, give rise to the β-amyloid peptide fragment. Both enzymes have now been molecularly cloned, and characterized to differing levels.

Several lines of evidence indicate that progressive cerebral deposition of β-amyloid peptide plays a seminal role in the pathogenesis of AD and can precede cognitive symptoms by years or decades. See, for example, Selkoe, Neuron, 6:487-498 (1991). The most important line of evidence is the discovery that missense DNA mutations at amino acid 717 of the 770-amino acid isoform of APP can be found in affected members but not unaffected members of several families with a genetically determined (familial) form of AD (Goate et al., Nature, 349:704-706 (1990); Chartier Harlan et al., Nature, 353:844-846 (1989); and Murrell et al., Science, 254:97-99 (1991.) Another such mutation, known as the Swedish variant, is comprised of a double mutation changing lysine⁵⁹⁵-methionine⁵⁹⁶ to asparagine⁵⁹⁵-leucine⁵⁹⁶ (with reference to the 695 isoform was found in a Swedish family) was reported in 1992 (Mullan et al., Nature Genet., 1:345-347 (1992). Genetic linkage analyses have demonstrated that these mutations, as well as certain other mutations in the APP gene, are the specific molecular cause of AD in the affected members of such families. In addition, a mutation at amino acid 693 of the 770-amino acid isoform of APP has been identified as the cause of the β-amyloid peptide deposition disease, HCHWA-D, and a change from alanine to glycine at amino acid 692 appears to cause a phenotype that resembles AD is some patients but HCHWA-D in others. The discovery of these and other mutations in APP in genetically based cases of AD prove that alteration of APP metabolism, and subsequent deposition of its β-amyloid peptide fragment, can cause AD.

Despite the progress which has been made in understanding the underlying mechanisms of AD and other β-amyloid peptide related diseases, there remains a need to develop methods and compositions for treatment of the disease(s). Ideally, the treatment methods would advantageously be based on drugs which are capable of inhibiting β-amyloid peptide release and/or its synthesis in vivo.

One approach toward inhibiting amyloid peptide synthesis in vivo is by inhibiting gamma secretase, the enzyme responsible for the carboxy-terminal cleavage resulting in production of β-amyloid peptide fragments of 40 or 42 residues in length. The immediate substrates for gamma secretase are β-cleaved, as well as α-cleaved carboxy-terminal fragments (CTF) of APP. The gamma-secretase cleavage site on β- and α-CTF fragments occurs in the predicted transmembrane domain of APP. Inhibitors of gamma-secretase have been demonstrated to effect amyloid pathology in transgenic mouse models (Dovey, H. F., V. John, J. P. Anderson, L. Z. Chen, P. de Saint Andrieu, L. Y. Fang, S. B. Freedman, B. Folmer, E. Goldbach, E. J. Holsztynska et al. (2001). “Functional gamma-secretase inhibitors reduce beta-amyloid peptide levels in brain.” J Neurochem 76(1): 173-81.)

Gamma secretase is recognized to be a multi-subunit complex comprised of the presenilins (PS1 or PS2), Nicastrin, Aph-1, and Pen 2 (De Strooper, B. (2003). “Aph-1, Pen-2, and Nicastrin with Presenilin generate an active gamma-Secretase complex.” Neuron 38(1): 9-12; Edbauer, D., E. Winkler, J. T. Regula, B. Pesold, H. Steiner and C. Haass (2003). “Reconstitution of gamma-secretase activity.” Nat Cell Biol 5(5): 486-8; Kimberly, W. T., M. J. LaVoie, B. L. Ostaszewski, W. Ye, M. S. Wolfe and D. J. Selkoe (2003). “Gamma-secretase is a membrane protein complex comprised of presenilin, nicastrin, Aph-1, and Pen-2.” Proc Natl Acad Sci USA 100(11): 6382-7). Much evidence indicates that PS comprises the catalytic moiety of the complex, while the other identified subunits are necessary for proper maturation and sub-cellular localization of the active enzyme complex (reviewed in De Strooper, B. (2003). “Aph-1, Pen-2, and Nicastrin with Presenilin generate an active gamma-Secretase complex.” Neuron 38(1): 9-12.) Consistent with this hypothesis: PS knock-out mice exhibit significant reductions in β-amyloid production (De Strooper, B., P. Saftig, K. Craessaerts, H. Vanderstichele, G. Guhde, W. Annaert, K. Von Figura and F. Van Leuven (1998). “Deficiency of presenilin-1 inhibits the normal cleavage of amyloid precursor protein.” Nature 391(6665): 387-90; Haass, C. and D. J. Selkoe (1998). “Alzheimer's disease. A technical KO of amyloid-beta peptide.” Nature 391(6665): 339-40; Herreman, A., L. Semeels, W. Annaert, D. Collen, L. Schoonjans and B. De Strooper (2000). “Total inactivation of gamma-secretase activity in presenilin-deficient embryonic stem cells.” Nat Cell Biol 2(7): 461-2); point mutations of putative active site aspartate residues in PS trans-membrane domains inhibit β-amyloid production in cells in a dominant negative fashion (Wolfe, M. S., W. Xia, B. L. Ostaszewski, T. S. Diehl, W. T. Kimberly and D. J. Selkoe (1999). “Two transmembrane aspartates in presenilin-1 required for presenilin endoproteolysis and gamma-secretase activity.” Nature 398(6727): 513-7; Kimberly, W. T., W. Xia, T. Rahmati, M. S. Wolfe and D. J. Selkoe (2000). “The transmembrane aspartates in presenilin 1 and 2 are obligatory for gamma-secretase activity and amyloid beta-protein generation.” J Biol Chem 275(5): 3173-8); active site directed substrate-based transition state isosteres designed to inhibit gamma secretase directly conjugate to PS (Esler, W. P., W. T. Kimberly, B. L. Ostaszewski, T. S. Diehl, C. L. Moore, J. Y. Tsai, T. Rahmati, W. Xia, D. J. Selkoe and M. S. Wolfe (2000). “Transition-state analogue inhibitors of gamma-secretase bind directly to presenilin-1.” Nat Cell Biol 2(7): 428-34; Li, Y. M., M. Xu, M. T. Lai, Q. Huang, J. L. Castro, J. DiMuzio-Mower, T. Harrison, C. Lellis, A. Nadin, J. G. Neduvelil et al. (2000). “Photoactivated gamma-secretase inhibitors directed to the active site covalently label presenilin 1.” Nature 405(6787): 689-94); finally, allosteric gamma secretase inhibitors have likewise been demonstrated to bind directly to PS (Seiffert, D., J. D. Bradley, C. M. Rominger, D. H. Rominger, F. Yang, J. E. Meredith, Jr., Q. Wang, A. H. Roach, L. A. Thompson, S. M. Spitz et al. (2000). “Presenilin-1 and -2 are molecular targets for gamma-secretase inhibitors.” J Biol Chem 275(44): 34086-91.)

Current evidence indicates that in addition to APP processing leading to β-amyloid synthesis, gamma-secretase also mediates the intra-membrane cleavage of other type I transmembrane proteins (reviewed in Fortini, M. E. (2002). “Gamma-secretase-mediated proteolysis in cell-surface-receptor signaling.” Nat Rev Mol Cell Biol 3(9): 673-84, see also Struhl, G. and A. Adachi (2000). “Requirements for presenilin-dependent cleavage of notch and other transmembrane proteins.” Mol Cell 6(3): 625-36.) Noteworthy among the known substrates of gamma-secretase is mammalian Notch 1. The Notch 1 protein is important for cell fate determination during development, and tissue homeostasis in the adult. Upon ligand engagement via the Notch ecto-domain, Notch undergoes sequential extra-cellular and intra-membrane processing analogous to APP. The intra-membrane processing of Notch mediated by gamma secretase leads to release of the Notch intracellular domain (NICD). The NICD fragment mediates Notch signaling via translocation to the nucleus, where it regulates expression of genes mediating cellular differentiation in many tissues during development, as well as in the adult.

Disruption of Notch signaling via genetic knock-out (KO) results in embryonic lethal phenotype in mice (Swiatek, P. J., C. E. Lindsell, F. F. del Amo, G. Weinmaster and T. Gridley (1994). “Notch1 is essential for postimplantation development in mice.” Genes Dev 8(6): 707-19; Conlon, R. A., A. G. Reaume and J. Rossant (1995). “Notch1 is required for the coordinate segmentation of somites.” Development 121(5): 1533-45.) The Notch KO phenotype is very similar to the phenotype observed PS1 KO mice, and precisely reproduced by PS1/PS2 double KO mice (De Strooper et al. (1998). “Deficiency of presenilin-1 inhibits the normal cleavage of amyloid precursor protein.” Nature 391(6665): 387-90; Donoviel, D. B., A. K. Hadjantonakis, M. Ikeda, H. Zheng, P. S. Hyslop and A. Bernstein (1999). “Mice lacking both presenilin genes exhibit early embryonic patterning defects.” Genes Dev 13(21): 2801-10; Herreman, A., L. Serneels, W. Annaert, D. Collen, L. Schoonjans and B. De Strooper (2000). “Total inactivation of gamma-secretase activity in presenilin-deficient embryonic stem cells.” Nat Cell Biol 2(7): 461-2.) This convergence of phenotypes observed in knock-out mice of either the substrate (Notch) or the enzyme (PS) suggests that inhibitors of gamma secretase that also inhibit Notch function may be limited as therapeutic agents owing to the importance of Notch function in adult tissues (Fortini, M. E. (2002). “Gamma-secretase-mediated proteolysis in cell-surface-receptor signaling.” Nat Rev Mol Cell Biol 3(9): 673-84.) As APP knock-out mice develop normally and without an overt phenotype Zheng, H., M. Jiang, M. E. Trumbauer, R. Hopkins, D. J. Sirinathsinghji, K. A. Stevens, M. W. Conner, H. H. Slunt, S. S. Sisodia, H. Y. Chen et al. (1996). “Mice deficient for the amyloid precursor protein gene.” Ann N Y Acad Sci 777: 421-6; Zheng, H., M. Jiang, M. E. Trumbauer, D. J. Sirinathsinghji, R. Hopkins, D. W. Smith, R. P. Heavens, G. R. Dawson, S. Boyce, M. W. Conner et al. (1995). “beta-Amyloid precursor protein-deficient mice show reactive gliosis and decreased locomotor activity.” Cell 81(4): 525-31, the cumulative evidence, therefore, suggests that preferred gamma secretase inhibitors would have selectivity for inhibiting gamma secretase processing of APP over gamma secretase processing of Notch.

### SUMMARY OF THE INVENTION

In a broad aspect, the invention provides compounds of Formula I: 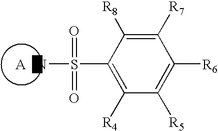
or pharmaceutically acceptable salts thereof, wherein
A-ring is selected from 1,2,3,4-tetrahydroisoquinolinyl, 1,2,3,4-tetrahydroquinolinyl, 1,2,3,4-tetrahydroquinoxalinyl, 1,2-dihydroquinolinyl, 1,3,4,6,7,8-Hexahydro-2H-pyrimido[1,2-a]pyrimidinyl, 1,3,8-triazaspiro[4.5]decan-4-onyl, 1,4,7-trioxa-10-azacyclododecanyl, 1,4-diazepanyl, 1H-naphtho[1,2-d]imidazolyl, 3,4-dihydro-2H-1,4-benzoxazinyl, azepanyl, decahydroisoquinolinyl, decahydroquinolinyl, indolinyl, octahydro-1H-indolyl, 3-azabicyclo[3.2.2]nonanyl, 1H-benzimidazolyl, indazolyl, indolyl, spiro[indene-1,4′-piperidinyl], 5H-dibenzo[b,f]azepinyl, 2-Hydroxymethyl-1,4-dioxa-8-azaspiro[4.5]decanyl, 10H-phenothiazinyl, 1,2,4,5-tetrahydrospiro[2-benzazepine-3,1′-cyclohexanyl], 2,3,4,9-tetrahydro-1H-β-carbolinyl, and 10,11-dihydro-5H-dibenzo[b,f]azepinyl, wherein each of the above groups is optionally substituted with 1, 2, 3 or 4 groups that are independently OH, H, CN, oxo, halo, C₁-C₆ alkoxy, C₁-C₆ alkyl, —C(O)NR₉R₁₀, —C(O)N(R₉)—C₁-C₆ alkyl-R₂₆, —S—C₁-C₆ alkyl, —C(O)R₂₈, C₂-C₆ alkenyl, —C(O)R₂₆, —C(O)R₂₇, C₀-C₆ alkyl-C(O)NR₉R₁₀, C₂-C₆ alkenyl-R₂₇, C₂-C₆ alkenyl-R₂₈, C₂-C₆ alkenyl-R₂₆, C₀-C₆ alkyl-R₂₇, C₀-C₆ alkyl-R₂₈, C₀-C₆ alkyl-R₂₆, C₀-C₆ alkyl-C(O)OR₁₁, C₀-C₆ alkyl-NR₉C(O)OR₁₁, NH₂, mono- or di-(C₁-C₆ alkyl)amino, —CF₃, —OCF₃, or NO₂; or
the A-ring is a group having the formula 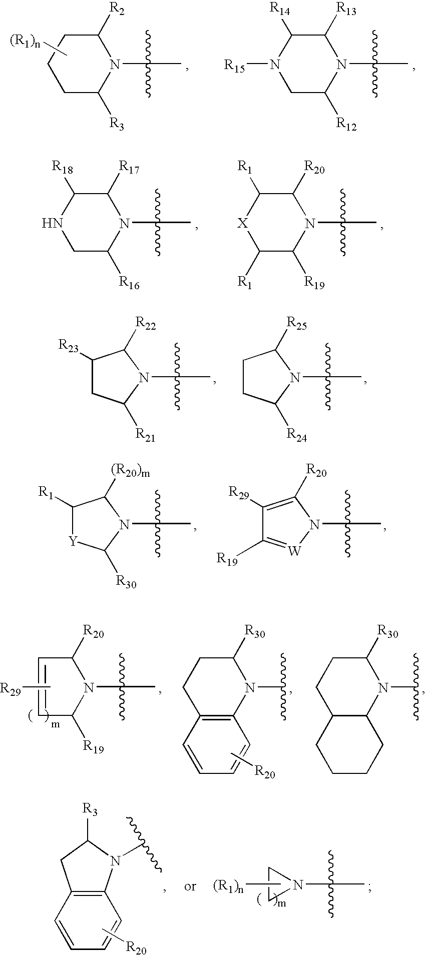
   wherein
W is CR₉ or nitrogen;
X is sulfur, SO₂, SO, or oxygen;
Y is sulfur, SO₂, SO, oxygen or NR₉;
m is 1 or 2;
n is 0 or an integer from 1 to 8;
R₁ at each occurrence is independently OH, H, CN, oxo, halo, C₁-C₆ alkyl, C₁-C₆ alkoxy, —C(O)R₁₁, C₂-C₆ alkenyl, —C(O)R₂₈, C(O)R₂₆, —C(O)R₂₇, C₂-C₆ alkenyl-R₂₇, C₂-C₆ alkenyl-R₂₈, C₂-C₆ alkenyl-R₂₆, C₀-C₆ alkyl-R₂₇, C₀-C₆ alkyl-R₂₈, C₀-C₆ alkyl-C(O)NR₉R₁₀, C₀-C₆ alkyl-C(O)OR₁₁, C₀-C₆ alkyl-NR₉C(O)OR₁₁, C₀-C₆ alkyl-R₂₆, C₀-C₆ alkyl-R₂₇, or C₀-C₆ alkyl-R₂₈, wherein the alkyl portions of each of the above are optionally substituted with 1, 2, 3, 4, 5 or 6 groups that are independently C₁-C₆ alkyl, hydroxy-C₁-C₆ alkyl, C₁-C₆ alkoxy, C₀-C₆ alkyl-R₂₆, halo, OH, oxo, CF₃, —OCF₃, NO₂, NH₂, mono- or di-(C₁-C₆ alkyl)amino, or CN;
R₂ and R₃ are independently H, oxo, —C(O)OR₁₁, mono- or di-(C₁-C₆ alkyl)amino, halo, C₁-C₆ alkyl, C₁-C₆ alkoxy, C₂-C₆ alkenyl, —C(O)R₂₈, —C(O)R₂₆, —C(O)R₂₇, C₂-C₆ alkenyl-R₂₇, C₂-C₆ alkenyl-R₂₈, C₂-C₆ alkenyl-R₂₆, C₀-C₆ alkyl-C(O)OR₁₁, C₀-C₆ alkyl-C(O)NR₉R₁₀, C₀-C₆ alkyl-R₂₆, C₀-C₆ alkyl-R₂₇, or C₀-C₆ alkyl-R₂₈, wherein the alkyl groups are optionally substituted with 1, 2, 3, 4, 5 or 6 groups that are independently C₁-C₆ alkyl, OH, C₁-C₆ alkoxy, halo, CF₃, —OCF₃, NO₂, NH₂, mono- or di-(C₁-C₆ alkyl)amino, or CN;
R₄, R₅, R₇ and R₈ are independently H, OH, NH₂, mono- or di-(C₁-C₆ alkyl)amino, halo, C₁-C₆ alkoxy, or C₁-C₆ alkyl, wherein the alkoxy and alkyl groups are optionally substituted with 1, 2, 3 or 4 that are independently halo, C₁-C₆ alkyl, C₁-C₆ alkoxy, OH, oxo, CF₃, —OCF₃, NO₂, NH₂, mono- or di-(C₁-C₆ alkyl)amino, or CN;
R₆ is chloro, fluoro, iodo, NO₂, CF₃, OCF₃ or CN;
R₉ and R₁₀ are independently H, C₁-C₆ alkyl, C₃-C₆ cycloalkyl, or C₀-C₆ alkyl-R₂₆, C₂-C₆ alkenyl, C₂-C₆ alkenyl-R₂₇, C₂-C₆ alkenyl-R₂₈, C₂-C₆ alkenyl-R₂₆, C₀-C₆ alkyl-R₂₇, C₀-C₆ alkyl-R₂₈; or
R₉ and R₁₀ together with the nitrogen to which they are attached form pyrrolidinyl, morpholino or piperidinyl;
R₁₁ is H, C₁-C₆ alkyl, C₁-C₆ cycloalkyl, C₂-C₆ alkenyl, C₂-C₆ alkenyl-R₂₇, C₂-C₆ alkenyl-R₂₈, C₂-C₆ alkenyl-R₂₆, C₀-C₆ alkyl-R₂₇, C₀-C₆ alkyl-R₂₈ or C₀-C₆ alkyl-R₂₆;
R₁₂ and R₁₃ are independently OH, H, CN, NH₂, —C(O)NR₉R₁₀, C₀-C₆ alkyl-R₂₆, C₀-C₆ alkyl-R₂₇, or C₀-C₆ alkyl-R₂₈, mono- or di-(C₁-C₆ alkyl)amino, halo, C₀-C₆ alkyl-C(O)OR₁₁, C₁-C₆ alkyl, or C₁-C₆ alkoxy;
R₁₄ is H, C₁-C₆ alkyl, or oxo;
R₁₅ is C₁-C₆ alkyl, C₁-C₆ alkoxy, hydroxy-C₁-C₆ alkyl, C₁-C₆ alkyl-O-(hydroxy-C₁-C₆ alkyl), C₂-C₆ alkenyl, —C(O)R₂₈, —C(O)R₂₆, C₂-C₆ alkenyl-R₂₇, C₂-C₆ alkenyl-R₂₈, C₂-C₆ alkenyl-R₂₆, C₀-C₆ alkyl-R₂₈, —CO—N(R₉)₂, —C(O)R₂₇, C₀-C₆ alkyl-C(O)R₂₈, —C(O)OR₁₁, C₀-C₆ alkyl-R₂₆, or C₀-C₆ alkyl-R₂₇, wherein the alkyl groups are optionally substituted with 1, 2, 3, 4, 5 or 6 groups that are independently C₁-C₆ alkyl, C₁-C₆ alkoxy, halo, OH, CF₃, —OCF₃, NO₂, NH₂, mono- or di-(C₁-C₆ alkyl)amino, or CN;
R₁₆ and R₁₇ are independently OH, H, CN, NH₂, mono- or di-(C₁-C₆ alkyl)amino, halo, C₂-C₆ alkenyl, —C(O)—R₂₈, —C(O)R₂₆, —C(O)R₂₇, C₂-C₆ alkenyl-R₂₇, C₂-C₆ alkenyl-R₂₈, C₂-C₆ alkenyl-R₂₆, —C(O)NR₉R₁₀, C₀-C₆ alkyl-R₂₆, C₀-C₆ alkyl-R₂₇, or C₀-C₆ alkyl-R₂₈, C₀-C₆ alkyl-C(O)OR₁₁, C₁-C₆ alkyl, or C₁-C₆ alkoxy;
R₁₈ is C₁-C₆ alkyl or oxo;
R₁₉ and R₂₀ are independently OH, H, CN, NH₂, mono- or di-(C₁-C₆ alkyl)amino, halo, C₂-C₆ alkenyl, —C(O)R₂₆, —C(O)R₂₇, —C(O)—R₂₈, C₂-C₆ alkenyl-R₂₇, C₂-C₆ alkenyl-R₂₈, C₂-C₆ alkenyl-R₂₆, C₁-C₆ alkyl, C₁-C₆ alkoxy, C₀-C₆ alkyl-C(O)OR₁₁, C₀-C₆—C(O)NR₉R₁₀, C₀-C₆ alkyl-R₂₆, C₀-C₆ alkyl-R₂₇, or C₀-C₆ alkyl-R₂₈, wherein the alkyl groups are optionally substituted with 1, 2, 3 or 4 groups that are independently C₁-C₆ alkyl, OH, C₁-C₆ alkoxy, halo, CF₃, —OCF₃, NO₂, NH₂, mono- or di-(C₁-C₆ alkyl)amino, or CN;
R₂₁, and R₂₂ are independently H, C₁-C₆ alkyl, C₁-C₆ alkoxy, C₁-C₆ alkyl-C₁-C₆ alkoxy, —C(O)OR₁₁, —C(O)NR₉R₁₀, hydroxy C₁-C₆ alkyl, C₀-C₆ alkyl-R₂₈, C₀-C₆ alkyl-R₂₇, C₀-C₆ alkyl-NR₉R₂₆, or —C(O)—O—C₀-C₆ alkyl-R₂₆;
R₂₃ is OH, CN, oxo, NH₂, mono- or di-(C₁-C₆ alkyl)amino, halo, C₁-C₆ alkyl, C₁-C₆ alkoxy, C₁-C₆ alkyl-NR₉R₂₆, C₁-C₆ alkyl-O—C₁-C₆ alkyl, —C(O)R₁₁, —C(O)R₂₇, —C(O)R₂₈, —C(O)NR₉R₁₀, —C(O)OR₁₁, C₀-C₆ alkyl-NR₉C(O)OR₁₁, C₀-C₆ alkyl-R₂₆, C₀-C₆ alkyl-R₂₇, or C₀-C₆ alkyl-R₂₈;
R₂₄ is H or C₁-C₆ alkyl;
R₂₅ is C₁-C₆ alkyl, C₂-C₆ alkenyl, C₂-C₆ alkenyl-R₂₇, C₂-C₆ alkenyl-R₂₈, C₂-C₆ alkenyl-R₂₆, C₀-C₆ alkyl-NR₉R₂₆, —C(O)O—C₀-C₆ alkyl-R₂₆ or C₀-C₆ alkyl-R₂₈, or C₀-C₆ alkyl-R₂₆ wherein the alkyl is optionally substituted with C₀-C₆ alkyl-R₂₆ or OH;
R₂₉ at each occurrence is independently OH, H, CN, halo, C₁-C₆ alkyl, C₁₁-C₆ alkoxy, C₂-C₆ alkenyl, —C(O)R₂₆, —C(O)R₂₇, —C(O)—R₂₈, C₂-C₆ alkenyl-R₂₇, C₂-C₆ alkenyl-R₂₈, C₂-C₆ alkenyl-R₂₆, —C(O)R₁₁, C₀-C₆ alkyl-C(O)NR₉R₁₀, —C(O)OR₁₁, C₀-C₆ alkyl-NR₉C(O)OR₁₁, C₀-C₆ alkyl-R₂₆, C₀-C₆ alkyl-R₂₇, or C₀-C₆ alkyl-R₂₈, wherein the alkyl portions of each of the above are optionally substituted with 1, 2, 3, 4, 5 or 6 groups that are independently C₁-C₆ alkyl, hydroxy-C₁-C₆ alkyl, C₁-C₆ alkoxy, C₀-C₆ alkyl-R₂₆, halo, OH, oxo, CF₃, —OCF₃, NO₂, NH₂, CN, mono- or di-(C₁-C₆ alkyl)amino;
R₃₀ is OH, H, oxo, CN, NH₂, mono- or di-(C₁-C₆ alkyl)amino, halo, C₁-C₆ alkyl, C₂-C₆ alkenyl, —C(O)—R₂₈, —C(O)R₂₆, —C(O)R₂₇, C₂-C₆ alkenyl-R₂₇, C₂-C₆ alkenyl-R₂₈, C₂-C₆ alkenyl-R₂₆, C₁-C₆ alkoxy, C₀-C₆ alkyl-C(O)OR₁₁, C₀-C₆ alkyl-C(O)NR₉R₁₀, C₀-C₆ alkyl-R₂₆, C₀-C₆ alkyl-R₂₇, or C₀-C₆ alkyl-R₂₈, wherein the alkyl groups are optionally substituted with 1, 2, 3 or 4 groups that are independently C₁-C₆ alkyl, OH, C₁-C₆ alkoxy, halo, CF₃, —OCF₃, NO₂, NH₂, mono- or di-(C₁-C₆ alkyl)amino, or CN;
R₂₆ is phenyl which is optionally substituted with 1, 2, 3, 4, or 5 groups that are independently C₁-C₆ alkyl, C₁-C₆ alkoxy, halo, OH, CF₃, —OCF₃, NO₂, NH₂, —C(O)N(R₉)₂, —NR₉C(O)N(R₉)₂, —NR₉C(O)OR₉, mono- or di-(C₁-C₆ alkyl)amino, or CN;
R₂₇ is pyridinyl, benzodioxolyl, quinolinyl, pyrimidinyl, furanyl, diazolyl, triazolyl, oxadiazolyl, oxazolyl, thiadiazolyl, 1,3-dihydro-2-oxo-benzoimidazol-1-yl, or benzoimidazolyl, each of which is optionally substituted with 1, 2, 3, 4, 5 or 6 groups that are independently C₁-C₆ alkyl, C₁-C₆ alkoxy, halo, OH, CF₃, —OCF₃, NO₂, NH₂, —C(O)N(R₉)₂, —NR₉C(O)N(R₉)₂, —NR₉C(O)OR₉, mono- or di-(C₁-C₆ alkyl)amino, or CN; and
R₂₈ is pyrrolidinyl, morpholino or piperidinyl, each of which is optionally substituted with 1, 2, 3, 4 or 5 groups that are independently C₁-C₆ alkyl, C₁-C₆ alkoxy, hydroxy-C₁-C₂ alkyl, halo, OH, CF₃, —OCF₃, NO₂, NH₂, —C(O)N(R₉)₂, —NR₉C(O)N(R₉)₂, —-NR₉C(O)OR₉, mono- or di-(C₁-C₆ alkyl)amino, or CN.

The compounds of Formula I inhibit β-amyloid peptide release and/or its synthesis and, therefore, are useful in the prevention of Alzheimer's Disease (AD) in patients susceptible to AD and/or in the treatment of patients with AD in order to inhibit further deterioration in their condition. The invention also, encompasses pharmaceutical compositions containing the compounds of Formula I, and methods employing such compounds or compositions in the treatment of cognitive diseases, including Alzheimer's disease.

The invention also provides a method of treating a patient who has, or in preventing a patient from getting, a disease or condition selected from the group consisting of Alzheimer's disease, for helping prevent or delay the onset of Alzheimer's disease, for treating patients with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, age related macular degeneration or diffuse Lewy body type of Alzheimer's disease and who is in need of such treatment which comprises administration of a therapeutically effective amount of a compound of formula (I).

In another aspect, the invention provides methods of preparing the compounds of interest, as well as intermediates useful in preparing the compounds of interest.

### DETAILED DESCRIPTION OF THE INVENTION

In embodiment 1, the invention provides for compounds according to Formula I.

In embodiment 2, the invention provides compounds of Formula I having the structure 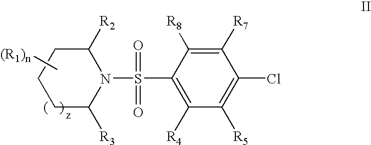
or pharmaceutically acceptable salts thereof, wherein
z is 0, 1, or 2;
n is 0, 1 or 2;
R₁ at each occurrence is independently OH, H, CN, oxo, halo, C₁-C₆ alkyl, C₂-C₆ alkenyl, —C(O)R₂₈, —C(O)R₂₆, —C(O)R₂₇, C₂-C₆ alkenyl-R₂₇, C₂-C₆ alkenyl-R₂₈, C₂-C₆ alkenyl-R₂₆, C₀-C₄ alkyl-R₂₈, C₀-C₄ alkyl-R₂₆, C₀-C₄ alkyl-R₂₇, —C(O)R₁₁, C₀-C₆ alkyl-C(O)NR₉R₁₀, —C(O)OR₁₁, or C₀-C₄ alkyl-NR₉C(O)OR₁₁, wherein each of the alkyl groups is optionally substituted with one or two groups that are independently OH or phenyl;
R₂ and R₃ are independently H, C₀-C₆ alkyl-C(O)NR₉R₁₀, C₀-C₆ alkyl-C(O)OR₁₁, C₂-C₆ alkenyl, —C(O)R₂₈, —C(O)R₂₆, —C(O)R₂₇, C₂-C₆ alkenyl-R₂₇, C₂-C₆ alkenyl-R₂₈, C₂-C₆ alkenyl-R₂₆, C₀-C₄ alkyl-R₂₆, C₀-C₄ alkyl-R₂₇, C₀-C₄ alkyl-R₂₈ or C₁-C₆ alkyl, wherein the alkyl group is optionally substituted with OH;
R₄, R₅, R₇ and R₈ are independently H or fluoro;
R₉ and R₁₀ are independently H, C₂-C₆ alkenyl, C₂-C₆ alkenyl-R₂₇, C₂-C₆ alkenyl-R₂₈, C₂-C₆ alkenyl-R₂₆, C₀-C₆ alkyl-R₂₇, C₀-C₆ alkyl-R₂₆, C₀-C₆ alkyl-R₂₈ or C₁-C₆ alkyl;
R₁ is H, C₂-C₆ alkenyl, C₂-C₆ alkenyl-R₂₇, C₂-C₆ alkenyl-R₂₈, C₂-C₆ alkenyl-R₂₆, C₀-C₆ alkyl-R₂₇, C₀-C₆ alkyl-R₂₆, C₀-C₆ alkyl-R₂₈ or C₁-C₆ alkyl;
R₂₆ is phenyl which is optionally substituted with 1, 2, 3, 4, or 5 groups that are independently C₁-C₆ alkyl, C₁-C₆ alkoxy, halo, OH, CF₃, —OCF₃, NO₂, NH₂, —C(O)N(R₉)₂, —NR₉C(O)N(R₉)₂, —NR₉C(O)OR₉, mono- or di-(C₁-C₆ alkyl)amino, or CN;
R₂₇ is pyridinyl, benzodioxolyl, quinolinyl, pyrimidinyl, furanyl, diazolyl, triazolyl, oxadiazolyl, oxazolyl, thiadiazolyl, 1,3-dihydro-2-oxo-benzoimidazol-1-yl, or benzoimidazolyl, each of which is optionally substituted with 1, 2, 3, 4, 5 or 6 groups that are independently C₁-C₆ alkyl, C₁-C₆ alkoxy, halo, OH, CF₃, —OCF₃, NO₂, NH₂, —C(O)N(R₉)₂, —NR₉C(O)N(R₉)₂, —NR₉C(O)OR₉, mono- or di-(C₁-C₆ alkyl)amino, or CN; and
R₂₈ is pyrrolidinyl, morpholino or piperidinyl, each of which is optionally substituted with 1, 2, 3, 4 or 5 groups that are independently C₁-C₆ alkyl, C₁-C₆ alkoxy, hydroxy-C₁-C₂ alkyl, halo, OH, CF₃, —OCF₃, NO₂, NH₂, —C(O)N(R₉)₂, —NR₉C(O)N(R₉)₂, —NR₉C(O)OR₉, mono- or di-(C₁-C₆ alkyl)amino, or CN.

In embodiment 2A, the invention provides compounds of Formula I having the structure 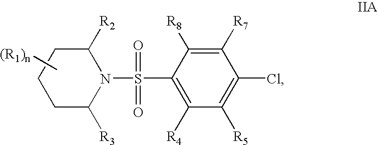
or pharmaceutically acceptable salts thereof, wherein
z is 0, 1, or 2;
R₁ at each occurrence is independently OH, H, CN, oxo, halo, C₁-C₆ alkyl, C₂-C₆ alkenyl, —C(O)R₂₈, —C(O)R₂₆, —C(O)R₂₇, C₂-C₆ alkenyl-R₂₇, C₂-C₆ alkenyl-R₂₈, C₂-C₆ alkenyl-R₂₆, C₀-C₄ alkyl-R₂₈, C₀-C₄ alkyl-R₂₆, C₀-C₄ alkyl-R₂₇, —C(O)R₁₁, C₀-C₆ alkyl-C(O)NR₉R₁₀, —C(O)OR₁₁, or C₀-C₄ alkyl-NR₉C(O)OR₁₁, wherein each of the alkyl groups is optionally substituted with one or two groups that are independently OH or phenyl;
R₂ and R₃ are independently H, C₀-C₆ alkyl-C(O)NR₉R₁₀, C₀-C₆ alkyl-C(O)OR₁₁, C₂-C₆ alkenyl, —C(O)R₂₈, —C(O)R₂₆, —C(O)R₂₇, C₂-C₆ alkenyl-R₂₇, C₂-C₆ alkenyl-R₂₈, C₂-C₆ alkenyl-R₂₆, C₀-C₄ alkyl-R₂₆, C₀-C₄ alkyl-R₂₇, C₀-C₄ alkyl-R₂₈ or C₁-C₆ alkyl, wherein the alkyl group is optionally substituted with OH;
R₄, R₅, R₇ and R₈ are independently H or fluoro;
R₉ and R₁₀ are independently H, C₂-C₆ alkenyl, C₂-C₆ alkenyl-R₂₇, C₂-C₆ alkenyl-R₂₈, C₂-C₆ alkenyl-R₂₆, C₀-C₆ alkyl-R₂₇, C₀-C₆ alkyl-R₂₆, C₀-C₆ alkyl-R₂₈ or C₁-C₆ alkyl;
R₁₁ is H, C₂-C₆ alkenyl, C₂-C₆ alkenyl-R₂₇, C₂-C₆ alkenyl-R₂₈, C₂-C₆ alkenyl-R₂₆, C₀-C₆ alkyl-R₂₇, C₀-C₆ alkyl-R₂₆, C₀-C₆ alkyl-R₂₈ or C₁-C₆ alkyl;
R₂₆ is phenyl which is optionally substituted with 1, 2, 3, 4, or 5 groups that are independently C₁-C₆ alkyl, C₁-C₆ alkoxy, halo, OH, CF₃, —OCF₃, NO₂, NH₂, —C(O)N(R₉)₂, —NR₉C(O)N(R₉)₂, —NR₉C(O)OR₉, mono- or di-(C₁-C₆ alkyl)amino, or CN;
R₂₇ is pyridinyl, benzodioxolyl, quinolinyl, pyrimidinyl, furanyl, diazolyl, triazolyl, oxadiazolyl, oxazolyl, thiadiazolyl, 1,3-dihydro-2-oxo-benzoimidazol-1-yl, or benzoimidazolyl, each of which is optionally substituted with 1, 2, 3, 4, 5 or 6 groups that are independently C₁-C₆ alkyl, C₁-C₆ alkoxy, halo, OH, CF₃, —OCF₃, NO₂, NH₂, —C(O)N(R₉)₂, —NR₉C(O)N(R₉)₂, —NR₉C(O)OR₉, mono- or di-(C₁-C₆ alkyl)amino, or CN; and
R₂₈ is pyrrolidinyl, morpholino or piperidinyl, each of which is optionally substituted with 1, 2, 3, 4 or 5 groups that are independently C₁-C₆ alkyl, C₁-C₆ alkoxy, hydroxy-C₁-C₂ alkyl, halo, OH, CF₃, —OCF₃, NO₂, NH₂, —C(O)N(R₉)₂, —NR₉C(O)N(R₉)₂, —NR₉C(O)OR₉, mono- or di-(C₁-C₆ alkyl)amino, or CN.

In embodiment 3, the invention provides compounds according to either embodiment 2 or 2A, wherein at least one of R₁, R₅, R₄, R₇, and R₈ is H, and R₂ and R₃ are independently H, R₂₇, or C₁-C₆ alkyl optionally substituted with OH. In one aspect, R₁, R₅, R₄, R₇, and R₈ are H. In other aspect, R₂ is H and R₃ is H, R₂₇, or C₁-C₆ alkyl optionally substituted with OH. In yet another aspect, R₃ is C₁-C₄-alkyl, wherein the C₁-C₄-alkyl is methyl or ethyl.

In embodiment 4, the invention provides compounds according to embodiment 3 wherein R₃ is C₁-C₄ alkyl substituted with OH. In one aspect, R₃ is hydroxymethyl.

In embodiment 5, the invention provides compounds according to embodiment 3 wherein R₃ is R₂₇. In one aspect, R₂₇ is pyridinyl, 1,3-dihydro-2-oxo-benzoimidazol-1-yl, benzodioxolyl, quinolinyl, pyrimidinyl, furanyl, or benzoimidazolyl. Preferably, R₃ is pyridinyl, quinolinyl, pyrimidinyl, or furanyl. More preferably, R₃ is pyridinyl.

In embodiment 6, the invention provides compounds according to embodiment 3 wherein R₂ and R₃ are independently C₁-C₆ alkyl. In one aspect, R₂ and R₃ are independently C₁-C₄ alkyl. In another aspect, R₂ and R₃ are methyl. In yet another aspect, the carbon atom to which one of the methyl groups is attached is in the R-configuration. In still another aspect, the carbon atom to which the other methyl group is attached is in the S-configuration.

In embodiment 7, the invention provides compounds according to embodiment 3 wherein R₂ and R₃ are H.

In embodiment 8, the invention provides compounds according to embodiment 2 or 2A, wherein at least one of R₄, R₅, R₈, R₇, R₂, and R₃ is H, n is 1 or 2, and R₁ is OH, halo, or C₁-C₆ alkyl optionally substituted with OH.

In embodiment 9, the invention provides compounds of embodiment 8 wherein R₄, R₅, R₈, R₇, R₂, and R₃ are H.

In embodiment 10, the invention provides compounds according to embodiment 9, wherein R₁ is C₁-C₆ alkyl optionally substituted with OH.

In embodiment 11, the invention provides compounds according to embodiment 10, wherein n is 1 or 2, and each R₁ is independently methyl or propyl . In another aspect, R₁ is methyl or propyl. In one aspect, R₁ is attached to the piperidinyl ring as

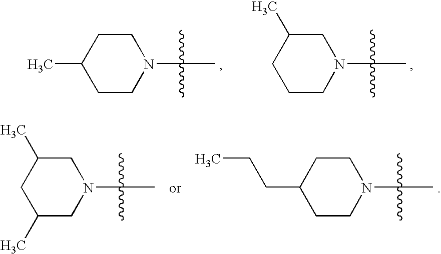

In embodiment 11a, the invention provides compounds of embodiment 3, wherein R₂, R₃ are independently H, or C₁-C₆ alkyl; and z is 2.

In embodiment 12, the invention provides compounds according to embodiment 10, wherein R₁ is hydroxymethyl or hydroxyethyl. In one aspect, R₁ is attached to the piperidinyl ring as

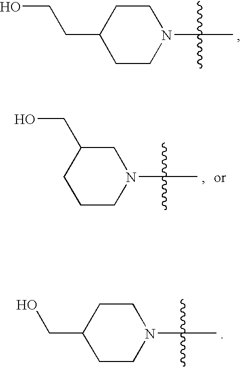

In embodiment 13, the invention provides compounds according to embodiment 9 wherein R₁ is halo. In one aspect, R₁ is bromo. In another aspect, R₁ is bromo and is attached to the piperidinyl ring as

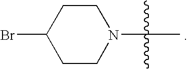

In embodiment 14, the invention provides compounds according to embodiment 9 wherein R₁ is OH. In one aspect, R₁ is OH and is attached to the piperidinyl ring as

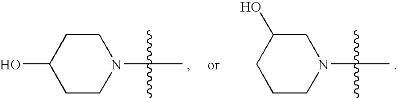

In embodiment 15, the invention provides compounds according to embodiment 2 or 2A, wherein at least one of R₄, R₅, R₈, and R₇ are H, R₂ and R₃ are independently H, C(O)NR₉R₁₀, or —C(O)OR₁₁, n is 1, and R₁ is —C(O)OR₁₁, C₁-C₄ alkyl-NR₉C(O)OR₁₁, or —C(O)NR₉R₁₀.

In embodiment 16, the invention provides compounds of embodiment 15 wherein R₂, R₃, R₄, R₅, R₇, and R₈ are H.

In embodiment 17, the invention provides compounds according to embodiment 16 wherein R₁ is —C(O)OR₁₁. In one aspect, R₁₁ is H or C₁-C₄ alkyl. In another aspect, R₁₁ is H. In yet another aspect, R₁ is —C(O)—OH and is attached to the piperidinyl ring as

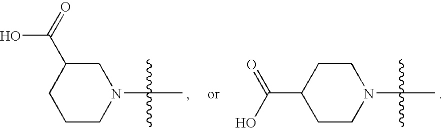

In embodiment 18, the invention provides compounds according to embodiment 17 wherein R₁₁ is C₁-C₄ alkyl. In one aspect, R₁₁ is —C₂H₅. In another aspect, R₁ is —C(O)OCH₂H₅ and is attached to the piperidinyl ring as

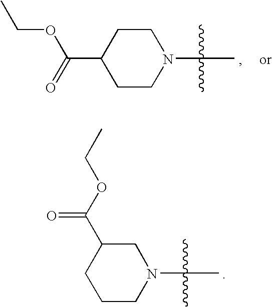

In embodiment 19, the invention provides compounds according to embodiment 16 wherein R₁ is C₁-C₄ alkyl-NR₉C(O)OR₁₁. In one aspect, R₉ and R₁₁ are independently H or C₁-C₄ alkyl. In another aspect, R₉ is H and R₁₁ is C₁-C₄ alkyl. In yet another aspect, R₉ is H and R₁₁ is tert-butyl. In still another aspect, R₁ is —C₂H₄—NHC(O)O-tert-butyl and is attached to the piperidinyl ring as

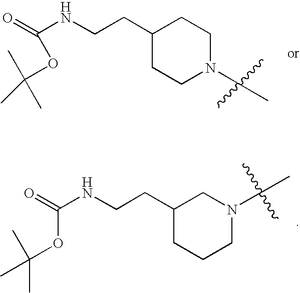

In embodiment 20, the invention provides compounds according to embodiment 16 wherein R₁ is —C(O)NR₉R₁₀. In one aspect, R₉ and R₁₀ are independently C₁-C₄ alkyl or H. In another aspect, R₉ and R₁₀ are C₁-C₄ alkyl. In yet another aspect, R₉ and R₁₀ are —C₂H₅. In still another aspect, R₁ is —C(O)N(C₂H₅)₂ and is attached to the piperidyl ring as

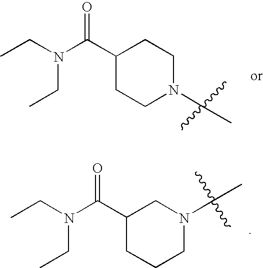

In embodiment 20a, the invention provides for compounds according to embodiment 15, wherein at least one of R₄, R₅, R₈, and R₇ are H, R₂ and R₃ are independently C(O)NR₉R₁₀ or —C(O)OR₁₁.

In embodiment 20b, the invention provides compounds according to embodiment 20a wherein R₄, R₅, R₇, and R₈ are H.

In embodiment 20c, the invention provides compounds according to embodiment 20b wherein one of R₂ and R₃ is C(O)NR₉R₁₀ and the other —C(O)OR₁₁. In one aspect, R₂ is the same as R₃. In another aspect, R₉, R₁₀ and R₁₁ are independently H or C₁-C₄ alkyl. In still another aspect R₉, R₁₀ and R₁₁ are H. In yet another aspect, R₉, R₁₀ and R₁₁ are C₁-C₄ alkyl.

In embodiment 21, the invention provides compounds according to embodiment 2 or 2A, wherein at least one of R₄, R₅, R₈, R₇, R₂, and R₃ is H, n is 1, and R₁ is R₂₇, or C₀-C₄ alkyl-piperidinyl wherein the piperidinyl portion is optionally substituted with hydroxy-C₁-C₄ alkyl, or R₁ is C₀-C₄ alkyl-R₂₆ wherein the alkyl portion is optionally substituted with phenyl and OH, and R₂₆ is phenyl.

In embodiment 22, the invention provides compounds according to embodiment 21 wherein R₄, R₅, R₈, R₇, R₂, and R₃ are H.

In embodiment 23, the invention provides compounds according to embodiment 22 wherein R₁ is C₀-C₄ alkyl-piperidinyl wherein the piperidinyl portion is substituted with hydroxy-C₁-C₄ alkyl. In one aspect, R₁ is piperidinyl or (hydroxyethyl)-piperidinylpropyl. In another aspect, R₁ is attached to the piperidinyl ring as

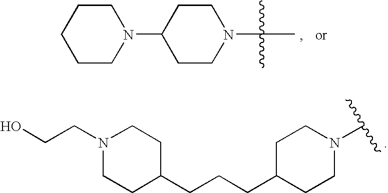

In embodiment 24, the invention provides compounds according to embodiment 22 wherein R₁ is C₀-C₄ alkyl-phenyl wherein the alkyl is optionally substituted with phenyl and OH. In one aspect, R₁ is phenyl, benzyl or 1,1-diphenyl-1-hydroxymethyl. In another aspect, R₁ is attached to the piperidinyl ring as

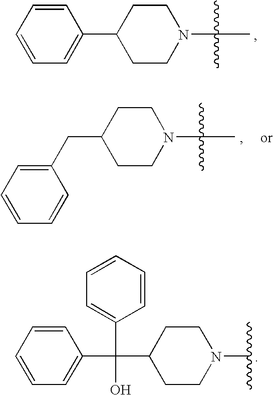

In embodiment 25, the invention provides compounds according to embodiment 22 wherein R₁ is R₂₇. In one aspect, wherein R₁ is pyridinyl, 1,3-dihydro-2-oxo-benzoimidazol-1-yl, benzodioxolyl, quinolinyl, pyrimidinyl, furanyl, or benzoimidazolyl. In another aspect, R₁ is 1,3-dihydro-2-oxo-benzoimidazol-1-yl. In yet another aspect, R₁ is attached to the piperidinyl ring as

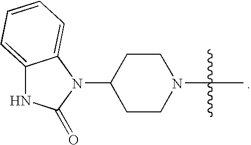

In embodiment 26, the invention provides compounds according to embodiment 2 or 2A, wherein at least one of R₄, R₅, R₈, R₇, R₂, and R₃ is H, n is 2, and R₁ at each occurrence is independently OH, CN, oxo, —C(O)R₁₁, —C(O)OR₁₁, —C(O)NR₉R₁₀, C₁-C₆ alkyl, or C₀-C₄ alkyl-R₂₆ wherein R₂₆ is phenyl optionally substituted with halo.

In embodiment 27, the invention provides compounds according to embodiment 26 wherein R₄, R₅, R₈, R₇, R₂, and R₃ are H.

In embodiment 28, the invention provides compounds according to embodiment 27 wherein one R₁ is OH and the other R₁ is C₀-C₄ alkyl-R₂₆. In one aspect, R₂₆ is phenyl optionally substituted with chloro or fluoro. In another aspect, R₂₆ is phenyl substituted with chloro. In yet another aspect, R₁ is phenyl or 4-chlorophenyl. In still another aspect, R₁ is attached to the piperidinyl ring as

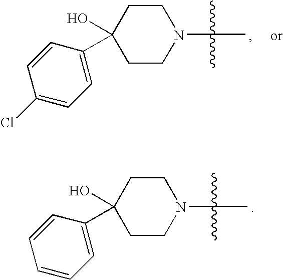

In embodiment 29, the invention provides compounds according to embodiment 27 wherein one R₁ is phenyl and the other R₁ is CN, —C(O)R₁₁, or C₁-C₆ alkyl. In one aspect, both R₁ groups are attached to the same carbon atom. In another aspect, the other R₁ is C₁-C₆ alkyl. In yet another aspect, the other R₁ is methyl. In still another aspect, R₁ is attached to the piperidinyl ring as

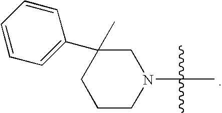

In embodiment 30, the invention provides compounds according to embodiment 29 wherein the other R₁ is —C(O)R₁₁. In one aspect, R₁₁, is H or C₁-C₄-alkyl. In another aspect, R₁₁ is C₁-C₄-alkyl. In yet another aspect, R₁₁ is methyl. In still another aspect, R₁ is attached to the piperidinyl ring as

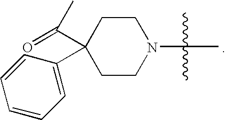

In embodiment 31, the invention provides compounds according to embodiment 29 wherein the other R₁ is CN. In one aspect, R₁ is attached to the piperidinyl ring as

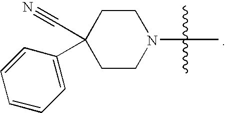

In embodiment 32, the invention provides compounds according to embodiment 27 wherein one R₁ is oxo. In one aspect, the other R₁ is —C(O)OR₁₁. In another aspect, R₁₁ is H or C₁-C₄ alkyl. In yet another aspect, R₁₁ is C₁-C₄-alkyl. In still another aspect, R₁₁ is ethyl. In still another aspect, R₁ is oxo and the other R₁ is —C(O)O—C₂H₅. In still another aspect, both R₁ are attached to the piperidinyl ring as

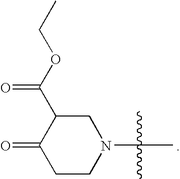

In embodiment 32a, the invention provides compounds according to embodiment 27 wherein one R₁ is oxo. In one aspect, the other R₁ is —C(O)NR₉R₁₀. In another aspect, R₉ and R₁₀ are independently H or C₁-C₄ alkyl. In yet another aspect, R₉ is C₁-C₄-alkyl and R₁₀ is H. In still another aspect, R₉ is ethyl. In still another aspect, R₁ is oxo and the other R₁ is —C(O)NH—C₂H₅. In still another aspect, both R₁ are attached to the piperidinyl ring as

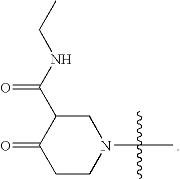

In embodiment 33, the invention provides compounds according to Formula I having the structure 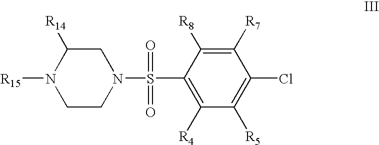
or pharmaceutically acceptable salts thereof, wherein
R₄, R₅, R₇ and R₈ are independently H or fluoro;
R₁₁ is C₁-C₄ alkyl, C₂-C₆ alkenyl, C₂-C₆ alkenyl-R₂₇, C₂-C₆ alkenyl-R₂₈, C₂-C₆ alkenyl-R₂₆, C₀-C₆ alkyl-R₂₇, C₀-C₆ alkyl-R₂₈ or C₀-C₄ alkyl-R₂₆;
R₉ is H, C₁-C₆ alkyl, C₃-C₆ cycloalkyl, or C₀-C₆ alkyl-R₂₆, C₂-C₆ alkenyl, C₂-C₆ alkenyl-R₂₇, C₂-C₆ alkenyl-R₂₈, C₂-C₆ alkenyl-R₂₆, C₀-C₆ alkyl-R₂₇, C₀-C₆ alkyl-R₂₈;
R₁₄ is H or C₁-C₆ alkyl;
R₁₅ is C₁-C₄ alkyl, hydroxy-C₁-C₄ alkyl, —CO—N(R₉)₂, C₁-C₄ alkyl-O-(hydroxy-C₁-C₄ alkyl), —C(O)R₂₇, C₂-C₆ alkenyl, —C(O)R₂₈, —C(O)R₂₆, C₂-C₆ alkenyl-R₂₇, C₂-C₆ alkenyl-R₂₈, C₂-C₆ alkenyl-R₂₆, C₀-C₆ alkyl-R₂₈, C₀-C₄ alkyl-C(O)R₂₈, —C(O)OR₁₁, C₀-C₄ alkyl-R₂₆, C₀-C₄ alkyl-R₂₇, wherein the alkyl groups are optionally substituted with one or more groups that are independently phenyl or methyl;
R₂₆ is phenyl which is optionally substituted with one or two groups that are independently C₁-C₄ alkyl, C₁-C₄ alkoxy, halo, CN, or CF₃;
R₂₇ is pyridinyl, diazolyl, triazolyl, oxadiazolyl, oxazolyl, thiadiazolyl, benzodioxolyl, quinolinyl, pyrimidinyl, or furanyl, each of which is optionally substituted with CF₃; and
R₂₈ is pyrrolidinyl, morpholino or piperidinyl which is optionally substituted with one or more groups that are independently C₁-C₄ alkyl, C₁-C₄ alkoxy, halo, CN, OH, CF₃, —OCF₃, NO₂, NH₂, mono- or di-(C₁-C₆ alkyl)amino.

In embodiment 34, the invention provides compounds according to embodiment 33 wherein any one of R₄, R₅, R₈, R₇, and R₁₄ is H, and R₁₅ is C₀-C₂ alkyl-phenyl wherein the alkyl is optionally substituted with methyl or phenyl, wherein the phenyl portion is optionally substituted with one or two groups selected from C₁-C₄ alkyl, halo, C₁-C₄ alkoxy, CF₃, or CN.

In embodiment 35, the invention provides compounds of embodiment 34 wherein R₄, R₅, R₈, R₇, and R₁₄ are H.

In embodiment 36, the invention provides compounds according to embodiment 35 wherein R₁₅ is C₁-C₂ alkyl-phenyl wherein the phenyl is optionally substituted with halo. In one aspect, R₁₅ is phenethyl or benzyl. In another aspect, R₁₅ is benzyl. In yet another aspect, the benzyl is substituted on the phenyl portion with chloro. In still another aspect, R₁₅ is 4-chlorobenzyl.

In embodiment 37, the invention provides compounds according to embodiment 35 wherein R₁₅ is C₁-C₂ alkyl-phenyl wherein the alkyl portion is substituted with methyl or phenyl. In one aspect, R₁₅ is —CH₂-phenyl, wherein the —CH₂— group is substituted with methyl or phenyl. In another aspect, R₁₅ is

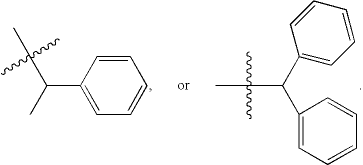

In embodiment 38, the invention provides compounds according to embodiment 35 wherein R₁₅ is phenyl substituted with one or two groups that are independently chloro, fluoro, methoxy, methyl, CN, or CF₃. In one aspect, R₁₅ is substituted with one or two chloro groups. In another aspect, R₁₅ is 4-chlorophenyl or 3-chlorophenyl. In yet another aspect, R₁₅ is 3,4-dichorophenyl or 3,5-dichlorophenyl. In still another aspect, R₁₅ is 2-methoxyphenyl. In still another aspect, R₁₅ is 4-fluorophenyl. In still another aspect, R₁₅ is 3-trifluoromethylphenyl. In still another aspect R₁₅ is 2-cyanophenyl. In still another aspect, R₁₅ is substituted with one or two methyl groups. In still another aspect, R₁₅ is 2-methylphenyl or 4-methylphenyl. In still another aspect, R₁₅ is 2,3-dimethylphenyl.

In embodiment 39, the invention provides compounds according to embodiment 33 wherein at least one of R₄, R₅, R₈, and R₇ is H, R₁₄ is methyl, and R₁₅ is C₀-C₂ alkyl-phenyl wherein phenyl portion is optionally substituted with C₁-C₄ alkyl or C₁-C₄ alkoxy.

In embodiment 40, the invention provides compounds according to embodiment 39 wherein R₄, R₅, R₈, and R₇ are H.

In embodiment 41, the invention provides compounds according to embodiment 40 wherein R₁₅ is phenyl.

In embodiment 42, the invention provides compounds according to embodiment 40 wherein R₁₅ phenyl substituted with C₁-C₄ alkyl or C₁-C₄ alkoxy. In one aspect, R₁₅ is phenyl substituted with C₁-C₄ alkyl. In another aspect, R₁₅ is phenyl substituted with methyl. In yet another aspect, R₁₅ is 4-methylphenyl or 3-methylphenyl. In still another aspect, R₁₅ is phenyl substituted with C₁-C₄ alkoxy. In still another aspect, R₁₅ is phenyl is substituted with methoxy. In still another aspect, R₁₅ is 4-methoxyphenyl.

In embodiment 43, the invention provides compounds according to embodiment 33 wherein at least one of R₄, R₅, R₈, R₇, and R₁₄ is H, and R₁₅ is C₀-C₂ alkyl-R₂₇, wherein R₂₇ is benzodioxolyl, pyrimidinyl, pyridinyl or quinolinyl, each of which is optionally substituted with CF₃.

In embodiment 44, the invention provides compounds of embodiment 43 wherein R₄, R₅, R₈, R₇, and R₁₄ are H.

In embodiment 45, the invention provides compounds according to embodiment 44 wherein R₁₅ is pyrimidinyl or pyridinyl. In one aspect, R₁₅ is pyrimidin-2-yl, pyridin-4-yl or pyridin-2-yl.

In embodiment 46, the invention provides compounds according to embodiment 44 wherein R₁₅ —CH₂—R₂₇. In one aspect, R₁₅ is benzodioxolylmethyl. In another aspect, the benzodioxolylmethyl is attached to the piperazinyl ring as

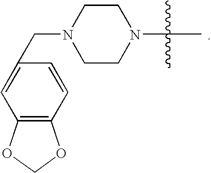

In embodiment 47, the invention provides compounds of embodiment 44 wherein R₁₅ is R₂₇ substituted with CF₃. In one aspect, R₁₅ is trifluoromethylpyridinyl or trifluoromethylquinolinyl. In another aspect, R₁₅ is 5-trifluoromethylpyridin-2-yl or 2-trifluoromethylquinolin-4-yl.

In embodiment 48, the invention provides compounds according to embodiment 33 wherein at least one of R₄, R₅, R₈, R₇, and R₁₄ is H, and R₁₅ is —C(O)—R₂₇, C₀-C₄ alkyl-C(O)-pyrrolidinyl, or —C(O)—OR₁₁.

In embodiment 49, the invention provides compounds according to embodiment 48 wherein R₄, R₅, R₈, R₇, and R₁₄ are H.

In embodiment 50, the invention provides compounds according to embodiment 49 wherein R₁₅ is —C(O)—OR₁₁. In one aspect, R₁₁ is C₂-C₄ alkyl or benzyl. In another aspect, R₁₁ is C₂-C₄ alkyl. In yet another aspect, R₁₁ is -tert-butyl or ethyl. In still another aspect, R₁₁ is benzyl.

In embodiment 51, the invention provides compounds according to embodiment 49 wherein R₁₅ is —C(O)—R₂₇. In one aspect, R₂₇ is pyridinyl, benzodioxolyl, quinolinyl, pyrimidinyl, or furanyl.

In embodiment 52, the invention provides compounds according to embodiment 49 wherein R₁₅ is C₀-C₄ alkyl-C(O)-pyrrolidinyl. In one aspect, R₁₅ is —CH₂—C(O)-pyrrolidinyl.

In embodiment 53, the invention provides compounds according to embodiment 33, wherein at least one of R₄, R₅, R₈, R₇, and R₁₄ is H, and R₁₅ is hydroxy-C₁-C₄ alkyl, C₁-C₄ alkyl-O-(hydroxy-C₁-C₄ alkyl), or C₁-C₄ alkyl.

In embodiment 54, the invention provides compounds of embodiment 53 wherein R₄, R₅, R₈, R₇, and R₁₄ are H.

In embodiment 55, the invention provides compounds according to embodiment 54 wherein R₁₅ is C₁-C₄ alkyl. In one aspect, R₁₅ is ethyl.

In embodiment 55, the invention provides compounds according to embodiment 54 wherein R₁₅ is hydroxy-C₁-C₄ alkyl. In one aspect, R₁₅ is hydroxyethyl.

In embodiment 56, the invention provides compounds according to embodiment 54 wherein R₁₅ is C₁-C₄ alkyl-O-(hydroxy-C₁-C₄ alkyl). In one aspect, R₁₅ is —C₂H₄—O—C₂H₄OH.

In embodiment 57, the invention provides compounds according to Formula I having the structure 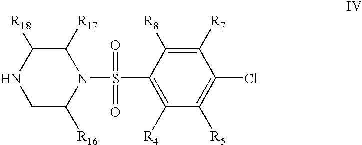
or pharmaceutically acceptable salts thereof, wherein
R₄, R₅, R₇ and R₈ are H or fluoro;
R₁₁, R₁₀ and R₉ are independently H or C₁-C₄ alkyl;
R₁₆ and R₁₇ are independently H, C₂-C₆ alkenyl, —C(O)—R₂₈, —C(O)R₂₆, —C(O)R₂₇, C₂-C₆ alkenyl-R₂₇, C₂-C₆ alkenyl-R₂₈, C₂-C₆ alkenyl-R₂₆, —C(O)NR₉R₁₀, —C(O)OR₁₁, C₀-C₄ alkyl-R₂₆, C₀-C₄ alkyl-R₂₇, C₀-C₄ alkyl-R₂₈, or C₀-C₄ alkyl-C(O)—OR₁₁;
R₁₈ is C₁-C₄ alkyl or oxo;
R₂₆ is phenyl which is optionally substituted with one or two groups that are independently C₁-C₄ alkyl, C₁-C₄ alkoxy, halo, CN, or CF₃;
R₂₇ is pyridinyl, diazolyl, triazolyl, oxadiazolyl, oxazolyl, thiadiazolyl, benzodioxolyl, quinolinyl, pyrimidinyl, or furanyl, each of which is optionally substituted with CF₃; and
R₂₈ is pyrrolidinyl, morpholino or piperidinyl which is optionally substituted with one or more groups that are independently C₁-C₄ alkyl, C₁-C₄ alkoxy, halo, CN, OH, CF₃, —OCF₃, NO₂, NH₂, mono- or di-(C₁-C₆ alkyl)amino.In embodiment 58, the invention provides compounds according to embodiment 57 wherein at least one of R₄, R₅, R₈, R₇, R₁₆, and R₁₇ is H, and R₁₈ is C₁-C₄ alkyl.

In embodiment 59, the invention provides compounds according to embodiment 58 wherein R₄, R₅, R₈, R₇, R₁₆, and R₁₇ are H.

In embodiment 60, the invention provides compounds according to embodiment 59 wherein R₁₈ is methyl. In one aspect, the atom to which the methyl is attached is in the R-configuration. In another aspect, the atom to which the methyl is attached is in the S-configuration.

In embodiment 61, the invention provides compounds according to embodiment 57 wherein at least any one of R₄, R₅, R₈, R₇, and R₁₆ is H, R₁₈ is oxo, and R₁₇ is C₀-C₁ alkyl-C(O)—OR₁₁.

In embodiment 62, the invention provides compounds of embodiment 61 wherein R₄, R₅, R₈, R₇, and R₁₆ are H.

In embodiment 63, the invention provides compounds according to embodiment 62 wherein R₁₁ is H or C₁-C₄ alkyl. In one aspect, R₁₁ is —C₂H₅.

In embodiment 64, the invention provides compounds according to Formula I having the structure 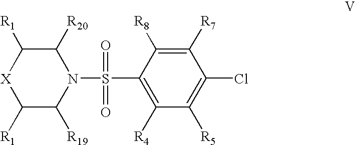
or pharmaceutically acceptable salts thereof, wherein
X is sulfur, SO₂, SO, or oxygen;
R₁ at each occurrence is independently H, C₁-C₄ alkyl, or C₁-C₄ alkoxy;
R₄, R₅, R₇ and R₈ are independently H or fluoro;
R₉, R₁₀ and R₁₁ are independently H or C₁-C₄-alkyl;
R₁₉ and R₂₀ are independently H, C₁-C₄ alkyl, C₂-C₆ alkenyl, —C(O)R₂₆, —C(O)R₂₇, —C(O)—R₂₈, C₂-C₆ alkenyl-R₂₇, C₂-C₆ alkenyl-R₂₈, C₂-C₆ alkenyl-R₂₆, —C(O)NR₉R₁₀, C₀-C₄ alkyl-R₂₆, C₀-C₄ alkyl-R₂₇, C₀-C₄ alkyl-R₂₈, or C₀-C₄ alkyl-C(O)—OR₁₁, or C₁-C₄ alkoxy;
R₂₆ is phenyl which is optionally substituted with one or two groups that are independently C₁-C₄ alkyl, C₁-C₄ alkoxy, halo, CN, or CF₃;
R₂₇ is pyridinyl, diazolyl, triazolyl, oxadiazolyl, oxazolyl, thiadiazolyl, benzodioxolyl, quinolinyl, pyrimidinyl, or furanyl, each of which is optionally substituted with CF₃; and
R₂₈ is pyrrolidinyl, morpholino or piperidinyl which is optionally substituted with one or more groups that are independently C₁-C₄ alkyl, C₁-C₄ alkoxy, halo, CN, OH, CF₃, —OCF₃, NO₂, NH₂, mono- or di-(C₁-C₆ alkyl)amino.

In embodiment 65, the invention provides compounds according to embodiment 64 wherein at least one of R₄, R₅, R₈, R₇, R₁₉, and R₂₀ is H, X is oxygen, and R₁ is H or C₁-C₄ alkyl.

In embodiment 66, the invention provides compounds according to embodiment 65 wherein R₄, R₅, R₈, R₇, R₁₉, and R₂₀ are H.

In embodiment 67, the invention provides compounds according to embodiment 66 wherein R₁ is C₁-C₄ alkyl. In one aspect, R₁ is methyl.

In embodiment 68, the invention provides compounds according to embodiment 66 wherein R₁ is H.

In embodiment 69, the invention provides compounds according to embodiment 64 wherein at least one of R₁, R₄, R₅, R₈, R₇, R₁₉, and R₂₀ is H, and X is sulfur.

In embodiment 70, the invention provides compounds according to embodiment 69 wherein R₁, R₄, R₅, R₈, R₇, R₁₉, and R₂₀ are H.

In embodiment 71, the invention provides compounds according to embodiment 64 wherein at least one of R₁, R₄, R₅, R₈, R₇, R₁₉, and R₂₀ is H, and X is SO₂.

In embodiment 72, the invention provides compounds according to embodiment 71 wherein R₁, R₄, R₅, R₈, R₇, R₁₉, and R₂₀ are H.

In embodiment 73, the invention provides compounds according to embodiment 64 wherein at least one of R₁, R₄, R₅, R₈, R₇, R₁₉, and R₂₀ is H, and X is SO.

In embodiment 74, the invention provides compounds according to embodiment 73 wherein R₁, R₄, R₅, R₈, R₇, R₁₉, and R₂₀ are H.

In embodiment 75, the invention provides compounds according to Formula I having the structure 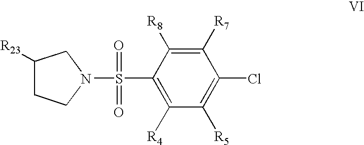
or pharmaceutically acceptable salts thereof, wherein
R₄, R₅, R₇ and R₈ are independently H of fluoro;
R₂₃ is OH, or —NR₉C(O)OR₁₁;
R₉ and R₁₁ are independently H or C₁-C₄ alkyl.

In embodiment 76, the invention provides compounds according to embodiment 75 wherein at least one of R₄, R₅, R₈, and R₇ is H, and R₂₃ is OH. In one aspect, R₄, R₅, R₈, and R₇ are H. In another aspect, the atom to which the OH group is attached is in the S-configuration. In yet another aspect, the atom to which the OH group is attached is in the R-configuration.

In embodiment 77, the invention provides compounds according to embodiment 75 wherein at least one of R₄, R₅, R₈, and R₇ is H, and R₂₃ is —NR₉—C(O)OR₁₁. In one aspect, R₄, R₅, R₈, and R₇ are H. In another aspect, R₉ and R₁₁ are independently H or C₁-C₄ alkyl. In yet another aspect, R₉ is H and R₁₁ is tert-butyl. In still another aspect, the atom to which R₂₃ is attached is in the S-configuration. In still another aspect, the atom to which R₂₃ is attached is in the R-configuration.

In embodiment 78, the invention provides compounds according to Formula I having the structure 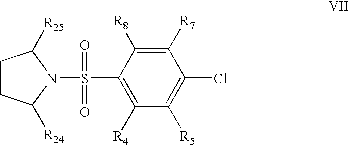
or pharmaceutically acceptable salts thereof, wherein
R₄, R₅, R₇ and R₈ are independently H or fluoro;
R₂₄ is H or C₁-C₄ alkyl; and
R₂₅ is C₁-C₄ alkyl, C₂-C₆ alkenyl, C₂-C₆ alkenyl-R₂₇, C₂-C₆ alkenyl-R₂₈, C₂-C₆ alkenyl-R₂₆, C₀-C₆ alkyl-R₂₈, C₀-C₄ alkyl-NH-phenyl, —C(O)O—C₀-C₄ alkyl-phenyl, C₀-C₄ alkyl-morpholinyl, C₀-C₄ alkyl-pyrrolidinyl, or C₀-C₄ alkyl-phenyl wherein the alkyl portion is optionally substituted with phenyl and OH;
R₂₆ is phenyl which is optionally substituted with one or two groups that are independently C₁-C₄ alkyl, C₁-C₄ alkoxy, halo, CN, or CF₃;
R₂₇ is pyridinyl, diazolyl, triazolyl, oxadiazolyl, oxazolyl, thiadiazolyl, benzodioxolyl, quinolinyl, pyrimidinyl, or furanyl, each of which is optionally substituted with CF₃; and
R₂₈ is pyrrolidinyl, morpholino or piperidinyl which is optionally substituted with one or more groups that are independently C₁-C₄ alkyl, C₁-C₄ alkoxy, halo, CN, OH, CF₃, —OCF₃, NO₂, NH₂, mono- or di-(C₁-C₆ alkyl)amino.

In embodiment 79, the invention provides compounds according to embodiment 78 wherein at least any one of R₄, R₅, R₈, and R₇ is H, R₂₄ and R₂₅ are independently H or C₁-C₄ alkyl.

In embodiment 80, the invention provides compounds according to embodiment 79 wherein R₄, R₅, R₈, and R₇ are H. In one aspect, R₂₄ and R₂₅ are C₁-C₄ alkyl. In another aspect, R₂₄ and R₂₅ are methyl. In yet another aspect, the atom to which R₂₄ is attached is in the S-configuration while the atom to which R₂₅ is attached is in the R-configuration. In still another aspect, the atom to which R₂₄ is attached is in the R-configuration while the atom to which R₂₅ is attached is in the S-configuration.

In embodiment 81, the invention provides compounds according to embodiment 78 wherein at least one of R₄, R₅, R₈, R₇, and R₂₄ is H, and R₂₅ is C₀-C₄ alkyl-NH-phenyl, C₀-C₄ alkyl-pyrrolidinyl, —C(O)O—C₀-C₄ alkyl-phenyl, or C₀-C₄ alkyl-phenyl wherein the alkyl is substituted with phenyl and OH.

In embodiment 82, the invention provides compounds according to embodiment 81 wherein R₄, R₅, R₈, R₇, and R₂₄ are H.

In embodiment 83, the invention provides compounds according to embodiment 82 wherein R₂₅ is C₀-C₄ alkyl-NH-phenyl. In one aspect, R₂₅ is —CH₂NH-phenyl. In another aspect, the atom to which R₂₅ is attached is in the R-configuration. In yet another aspect, the atom to which R₂₅ is attached is in the S-configuration.

In embodiment 84, the invention provides compounds according to embodiment 82 wherein R₂₅ is C₀-C₄ alkyl-pyrrolidinyl. In one aspect, R₂₅ is —CH₂-pyrrolidinyl. In another aspect, the atom to which R₂₅ is attached is in the S-configuration. In yet another aspect, the atom to which R₂₅ is attached is in the R-configuration.

In embodiment 85, the invention provides compounds according to embodiment 82 wherein R₂₅ is —C(O)O—C₀-C₄ alkyl-phenyl. In one aspect, R₂₅ is —C(O)—OCH₂-phenyl. In another aspect, the atom to which R₂₅ is attached is in the S-configuration. In yet another aspect, the atom to which R₂₅ is attached is in the R-configuration.

In embodiment 86, the invention provides compounds according to embodiment 82 wherein R₂₅ is C₀-C₄ alkyl-phenyl wherein the alkyl portion is substituted with phenyl and OH. In one aspect, R₂₅ is —C(OH)(phenyl)₂. In another aspect, the atom to which R₂₅ is attached is in the S-configuration. In yet another aspect, R₂₅ is attached is in the R-configuration.

In embodiment 87, the invention provides compounds according to Formula I having the structure 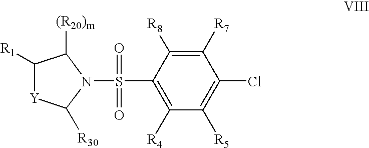
or pharmaceutically acceptable salts thereof, wherein
Y is sulfur, SO₂, SO, oxygen, or NR₉;
m is 1 or 2;
R₁ is H, oxo or C₁-C₆-alkyl;
R₄, R₅, R₇ and R₈ are independently H or fluoro;
R₉, R₁₀ and R₁₁ are independently H, C₁-C₄ alkyl, C₃-C₆ cycloalkyl, or C₀-C₆ alkyl-R₂₆;
R₃₀ is H, oxo, C₁-C₄ alkyl, C₂-C₆ alkenyl, —C(O)—R₂₈, —C(O)R₂₆, —C(O)R₂₇, C₂-C₆ alkenyl-R₂₇, C₂-C₆ alkenyl-R₂₈, C₂-C₆ alkenyl-R₂₆, —C(O)NR₉R₁₀, C₀-C₄ alkyl-R₂₆, C₀-C₄ alkyl-R₂₇, C₀-C₄ alkyl-R₂₈, or C₀-C₄ alkyl-C(O)—OR₁₁;
R₂₀ is H, C₁-C₄ alkyl, C₂-C₆ alkenyl, —C(O)—R₂₈, —C(O)R₂₆, —C(O)R₂₇, C₂-C₆ alkenyl-R₂₇, C₂-C₆ alkenyl-R₂₈, C₂-C₆ alkenyl-R₂₆, —C(O)NR₉R₁₀, C₀-C₄ alkyl-R₂₆, C₀-C₄ alkyl-R₂₇, C₀-C₄ alkyl-R₂₈, or C₀-C₄ alkyl-C(O)—OR₁₁;
R₂₆ is phenyl which is optionally substituted with one or two groups that are independently OH, C₁-C₄ alkyl, or C₁-C₄ alkoxy;
R₂₇ is pyridinyl, diazolyl, triazolyl, oxadiazolyl, oxazolyl, thiadiazolyl, benzodioxolyl, quinolinyl, pyrimidinyl, or furanyl, each of which is optionally substituted with CF₃; and
R₂₈ is pyrrolidinyl, morpholino or piperidinyl which is optionally substituted with one or more groups that are independently C₁-C₄ alkyl, C₁-C₄ alkoxy, halo, CN, OH, CF₃, —OCF₃, NO₂, NH₂, mono- or di-(C₁-C₆ alkyl)amino.

In embodiment 88, the invention provides compounds according to embodiment 87 wherein at least one of R₄, R₅, R₈, and R₇ is H, Y is NH, m is 2, R₁ and R₃₀ are oxo, and R₂₀ is phenyl optionally substituted with C₁-C₄ alkyl or OH.

In embodiment 89, the invention provides compounds according to embodiment 88 wherein R₄, R₅, R₈, and R₇ are H.

In embodiment 90, the invention provides compounds according to embodiment 89 wherein at least one R₂₀ is phenyl.

In embodiment 91, the invention provides compounds according to embodiment 89 wherein one R₂₀ is phenyl and the other is phenyl substituted with C₁-C₄ alkyl or OH. In one aspect, the other R₂₀ is phenyl substituted with the C₁-C₄ alkyl. In another aspect, the other R₂₀ is phenyl substituted with methyl. In still another aspect, the other R₂₀ is 4-methylphenyl. In still another aspect, the other R₂₀ is phenyl substituted with the OH. In still another aspect, the other R₂₀ is 3-hydroxyphenyl.

In embodiment 92, the invention provides compounds according to embodiment 87 wherein at least one of R₄, R₅, R₈, R₇, R₁, and R₃₀ is H, Y is oxygen, m is 2, and R₂₀ is C₁-C₄ alkyl.

In embodiment 93, the invention provides compounds according to embodiment 92 wherein R₄, R₅, R₈, R₇, R₁, and R₁₉ are H. In one aspect, R₂₀ is methyl.

In embodiment 94, the invention provides compounds according to embodiment 87 wherein at least one of R₁, R₅, R₄, R₈, and R₇ is H, Y is sulfur, m is 1, R₃₀ is R₂₆ wherein R₂₆ is phenyl substituted with two groups that are C₁-C₄ alkoxy, and R₂₀ is —C(O)NR₉R₁₀.

In embodiment 95, the invention provides compounds according to embodiment 94 wherein R₁, R₄, R₅, R₈, and R₇ are H. In one aspect, the carbon atom to which R₂₀ is attached is in the R-configuration. In another aspect, the carbon atom to which R₂₀ is attached is in the S-configuration.

In embodiment 96, the invention provides compounds according to embodiment 95 wherein R₃₀ is dimethoxyphenyl. In one aspect, R₃₀ is 3,4-dimethoxyphenyl.

In embodiment 97, the invention provides compounds according to embodiment 96 wherein R₉ is H and R₁₀ is C₁-C₄ alkyl-phenyl. In one aspect, R₁₀ is benzyl. In another aspect, R₁₀ is phenyl.

In embodiment 98, the invention provides compounds according to embodiment 96 wherein R₉ is H and R₁₀ is C₁-C₄ alkyl or C₃-C₆ cycloalkyl. In one aspect, R₁₀ is butyl or sec-butyl. In another aspect, R₁₀ is cyclohexyl.

In embodiment 99, the invention provides compounds according to embodiment 87 wherein at least one of R₄, R₅, R₈, R₇, R₁, R₂₀, and R₃₀ is H, and Y is sulfur.

In embodiment 100, the invention provides compounds according to embodiment 99 wherein R₄, R₅, R₈, R₇, R₁, R₂₀, and R₃₀ are H.

In embodiment 101, the invention provides compounds according to Formula I having the structure 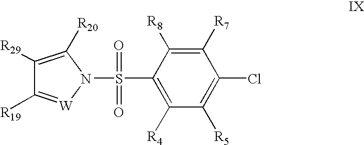
or pharmaceutically acceptable salts thereof, wherein
W is CR₉ or nitrogen;
R₂₉ is H or halo;
R₄, R₅, R₇ and R₈ are independently H or fluoro;
R₉, R₁₀ and R₁₁, are independently H or C₁-C₄-alkyl;
R₁₉ and R₂₀ are independently H, C₁-C₄ alkyl, C₂-C₆ alkenyl, —C(O)R₂₆, —C(O)R₂₇, —C(O)—R₂₈, C₂-C₆ alkenyl-R₂₇, C₂-C₆ alkenyl-R₂₈, C₂-C₆ alkenyl-R₂₆, —C(O)OH, C₀-C₆ alkyl-C(O)NR₉R₁₀, C₀-C₄ alkyl-R₂₆, C₀-C₄ alkyl-R₂₇, C₀-C₄ alkyl-R₂₈, or C₀-C₄ alkyl-C(O)—OR₁₁;
R₂₆ is phenyl which is optionally substituted with one or two groups that are independently OH or halo;
R₂₇ is pyridinyl, diazolyl, triazolyl, oxadiazolyl, oxazolyl, thiadiazolyl, benzodioxolyl, quinolinyl, pyrimidinyl, or furanyl, each of which is optionally substituted with CF₃; and
R₂₈ is pyrrolidinyl, morpholino or piperidinyl which is optionally substituted with one or more groups that are independently C₁-C₄ alkyl, C₁-C₄ alkoxy, halo, CN, OH, CF₃, —OCF₃, NO₂, NH₂, mono- or di-(C₁-C₆ alkyl)amino.

In embodiment 102, the invention provides compounds according to embodiment 101 wherein at least one of R₄, R₅, R₈, and R₇ is H, R₂₉ is H or halo, R₂₀ is H or —C(O)OH, and R₁₉ is H, C₁-C₄ alkyl, or R₂₆, wherein R₂₆ is phenyl substituted with chloro and OH.

In embodiment 103, the invention provides compounds according to embodiment 102 wherein R₄, R₅, R₈, and R₇ are H.

In embodiment 104, the invention provides compounds according to embodiment 103 wherein R₂₉ is halo, R₂₀ is —C(O)—OH, and R₁₉ is H. In one aspect, R₂₉ is iodo.

In embodiment 105, the invention provides compounds according to embodiment 103 wherein R₂₉ is halo, R₂₀ is H, and R₁₉ is C₁-C₄ alkyl. In one aspect, R₂₉ is bromo. In another aspect, R₁₉ is methyl.

In embodiment 106, the invention provides compounds according to embodiment 103 wherein R₂₉ and R₂₀ are H, and R₁₉ is phenyl substituted with chloro and OH. In one aspect, R₁₉ is 2-hydroxy-5-chloro-phenyl.

In embodiment 107, the invention provides compounds according to Formula I having any of the following structures 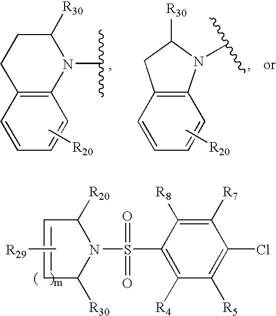
or pharmaceutically acceptable salts thereof, wherein
m is 1 or 2;
R₂₉ is H, C₁-C₆ alkyl, or C₁-C₆ alkoxy;
R₄, R₅, R₇ and R₈ are independently H or fluoro; and
R₉, R₁₀ and R₁₁, are independently H, C₁-C₄-alkyl, or C₀-C₆ alkyl-R₂₆;
R₃₀ and R₂₀ are independently H, C₁-C₄ alkyl, C₂-C₆ alkenyl, —C(O)—R₂₈, —C(O)R₂₆, —C(O)R₂₇, C₂-C₆ alkenyl-R₂₇, C₂-C₆ alkenyl-R₂₈, C₂-C₆ alkenyl-R₂₆, C₁-C₆ alkoxy, —C(O)NR₉R₁₀, C₀-C₄ alkyl-R₂₆, C₀-C₄ alkyl-R₂₇, C₀-C₄ alkyl-R₂₈, or C₀-C₄ alkyl-C(O)—OR₁₁;
R₂₆ is phenyl which is optionally substituted with one or two groups that are independently C₁-C₄ alkyl, C₁-C₄ alkoxy, halo, CN, or CF₃;
R₂₇ is pyridinyl, diazolyl, triazolyl, oxadiazolyl, oxazolyl, thiadiazolyl, benzodioxolyl, quinolinyl, pyrimidinyl, or furanyl, each of which is optionally substituted with CF₃; and
R₂₈ is pyrrolidinyl, morpholino or piperidinyl which is optionally substituted with one or more groups that are independently C₁-C₄ alkyl, C₁-C₄ alkoxy, halo, CN, OH, CF₃, —OCF₃, NO₂, NH₂, mono- or di-(C₁-C₆ alkyl)amino.

In embodiment 108, the invention provides compounds according to embodiment 107 wherein at least one of R₄, R₅, R₈, R₇ and R₂₉ is H, m is 1, and R₃₀ and R₂₀ are independently H or C₁-C₄ alkyl.

In embodiment 109, the invention provides compounds according to embodiment 108 wherein R₄, R₅, R₈, R₇ and R₂₉ are H.

In embodiment 110, the invention provides compounds according to embodiment 109 wherein R₃₀ and R₂₀ are C₁-C₄ alkyl. In one aspect, R₂₀ and R₃₀ are methyl.

In embodiment 111, the invention provides compounds according to embodiment 109 wherein R₃₀ and R₂₀ are H.
In embodiment 112, the invention provides compounds according to embodiment 107 wherein at least one of R₂₉, R₅, R₄, R₈, R₇, R₃₀, and R₂₀ is H, and m is 2. In one aspect, R₂₉, R₅, R₄, R₈, R₇, R₃₀, and R₂₀ are H.

In embodiment 112A, the invention provides compounds according to any one of embodiments 107, 108, 109, 110, 111, or 112 with the following core:

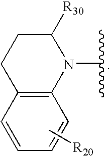

In embodiment 112B, the invention provides compounds according to any one of embodiments 107, 108, 109, 110, 111, or 112 with the following core:

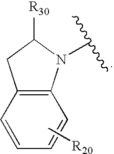

In embodiment 112B1, the invention provides compounds of embodiment 112B, wherein R₃₀ is C(O)NR₉R₁₀, where R₉ and R₁₀ are independently H, C₁-C₄-alkyl, or C₀-C₆ alkyl-R₂₆.

In another aspect, R₉ is H or methyl. In still another aspect, R₉ is H and R₁₀ is —CH₂—R₂₆.

In embodiment 112C, the invention provides compounds according to any one of embodiments 107, 108, 109, 110, 111, or 112 with the following core:

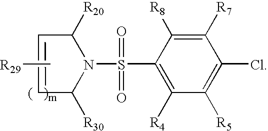

In embodiment 113, the invention provides compounds according to Formula I having the structure 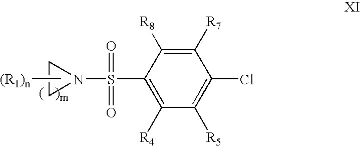
or pharmaceutically acceptable salts thereof, wherein
n is 0, or an interger from 1 to 3;
m is 1 or 2;
R₁ is H, C₁-C₆ alkyl, C₂-C₆ alkenyl, —C(O)R₂₈, —C(O)R₂₆, —C(O)R₂₇, C₂-C₆ alkenyl-R₂₇, C₂-C₆ alkenyl-R₂₈, C₂-C₆ alkenyl-R₂₆, C₀-C₆ alkyl-R₂₇, C₀-C₆ alkyl-R₂₈, C₀-C₆ alkyl-C(O)NR₉R₁₀, C₀-C₆ alkyl-C(O)OR₁₁, C₁-C₆ alkoxy, or C₀-C₄ alkyl-phenyl;
R₄, R₅, R₇ and R₈ are independently H or fluoro;
R₂₆ is phenyl which is optionally substituted with one or two groups that are independently C₁-C₄ alkyl, C₁-C₄ alkoxy, halo, CN, or CF₃;
R₂₇ is pyridinyl, diazolyl, triazolyl, oxadiazolyl, oxazolyl, thiadiazolyl, benzodioxolyl, quinolinyl, pyrimidinyl, or furanyl, each of which is optionally substituted with CF₃; and
R₂₈ is pyrrolidinyl, morpholino or piperidinyl which is optionally substituted with one or more groups that are independently C₁-C₄ alkyl, C₁-C₄ alkoxy, halo, CN, OH, CF₃, —OCF₃, NO₂, NH₂, mono- or di-(C₁-C₆ alkyl)amino.

In embodiment 114, the invention provides compounds according to embodiment 113 wherein at least one of R₄, R₅, R₈ and R₇ is H, m and n are 1, and R₁ is C₀-C₄ alkyl-phenyl.

In embodiment 115, the invention provides compounds according to embodiment 114 wherein R₄, R₅, R₈ and R₇ are H.

In embodiment 116, the invention provides compounds according to embodiment 115 wherein R₁ is phenyl.

In embodiment 117, the invention provides compounds according to embodiment 113 wherein at least one of R₁, R₅, R₄, R₈, and R₇ is H, and m is 2.

In embodiment 118, the invention provides compounds according to embodiment 117 wherein R₁, R₅, R₄, R₉ , and R₇ are H.

In embodiment 119, the invention provides a pharmaceutical composition comprising a compound of any one of the embodiments 1 to 118 and at least one pharmaceutically acceptable carrier, solvent, adjuvant or excipient, or a combination thereof.

In embodiment 120, the invention provides a method of treating a patient who has, or in preventing a patient from getting, a disease or condition selected from the group consisting of Alzheimer's disease (AD), for helping prevent or delay the onset of Alzheimer's disease, for treating patients with mild cognitive impairment (MCI) and preventing or delaying the onset of Alzheimer's disease in those who would progress from MCI to AD, for treating Down's syndrome, for treating humans who have Hereditary Cerebral Hemorrhage with Amyloidosis of the Dutch-Type, for treating cerebral amyloid angiopathy and preventing its potential consequences, i.e. single and recurrent lobar hemorrhages, for treating other degenerative dementias, including dementias of mixed vascular and degenerative origin, dementia associated with Parkinson's disease, dementia associated with progressive supranuclear palsy, dementia associated with cortical basal degeneration, age related macular degeneration, or diffuse Lewy body type of Alzheimer's disease and who is in need of such treatment which comprises administration of a therapeutically effective amount of a compounds of any one of embodiments 1 to 119.

The invention further provides for a method of treating a patient who has, or in preventing or delaying a patient from getting, a disease or condition selected from the group consisting of Alzheimer's disease (AD), mild cognitive impairment (MCI), Down's syndrome, Hereditary Cerebral Hemorrhage with Amyloidosis of the Dutch-Type, cerebral amyloid angiopathy and its potential consequences, i.e. single and recurrent lobar hemorrhages, other degenerative dementias, including dementias of mixed vascular and degenerative origin, dementia associated with Parkinson's disease, dementia associated with progressive supranuclear palsy, dementia associated with cortical basal degeneration, age related macular degeneration, or diffluse Lewy body type of Alzheimer's disease and who is in need of such treatment which comprises administration of a therapeutically effective amount of a compound or salt of formula I.

The invention also provides for a method of preparing a compound or salt of formula I.

In another aspect, the compounds of the invention have minimal interaction or preferably, no interaction with Notch.

Defintions

The definitions and explanations below are for the terms as used throughout this entire document including both the specification and the claims.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

Where multiple substituents are indicated as being attached to a structure, it is to be understood that the substituents can be the same or different. Thus for example “Rₘ optionally substituted with 1, 2 or 3 R_{q} groups” indicates that Rₘ is substituted with 1, 2, or 3 R_{q} groups where the R_{q} groups can be the same or different.

APP, amyloid precursor protein, is defined as any APP polypeptide, including APP variants, mutations, and isoforms, for example, as disclosed in U.S. Pat. No. 5,766,846. A beta, amyloid beta peptide, is defined as any peptide resulting from beta-secretase mediated cleavage of APP, including peptides of 39, 40, 41, 42, and 43 amino acids, and extending from the beta-secretase cleavage site to amino acids 39, 40, 41, 42, or 43.

Pharmaceutically acceptable refers to those properties and/or substances that are acceptable to the patient from a toxicological and/or safety point of view.

A therapeutically effective amount is defined as an amount effective to reduce or lessen at least one symptom of the disease being treated or to reduce or delay onset of one or more clinical markers or symptoms of the disease.

By “alkyl” and “C₁-C₆ alkyl” in the present invention is meant straight or branched chain alkyl groups having 1-6 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl. It is understood that in cases where an alkyl chain of a substituent (e.g. of an alkyl, alkoxy or alkenyl group) is shorter or longer than 6 carbons, it will be so indicated in the second “C” as, for example, “C₁-C₁₀ indicates a maximum of 10 carbons.

By “alkoxy” and “C₁-C₆ alkoxy” in the present invention is meant straight or branched chain alkyl groups having 1-6 carbon atoms, attached through at least one divalent oxygen atom, such as, for example, methoxy, ethoxy, propoxy, isopropoxy, n-butoxy, sec-butoxy, tert-butoxy, pentoxy, isopentoxy, neopentoxy, hexoxy, and 3-methylpentoxy.

By the term “halogen” in the present invention is meant fluorine, bromine, chlorine, and/or iodine.

“Alkenyl” and “C₂-C₆ alkenyl” means straight and branched hydrocarbon radicals having from 2 to 6 carbon atoms and from one to three double bonds and includes, for example, ethenyl, propenyl, 1-but-3-enyl, 1-pent-3-enyl, 1-hex-5-enyl and the like.

“Alkynyl” and “C₂-C₆ alkynyl” means straight and branched hydrocarbon radicals having from 2 to 6 carbon atoms and one or two triple bonds and includes ethynyl, propynyl, butynyl, pentyn-2-yl and the like.

As used herein, the term “cycloalkyl” refers to saturated carbocyclic radicals having three to twelve carbon atoms. The cycloalkyl can be monocyclic, a polycyclic fused system, or a bi or polycyclic bridged system, such as adamantyl or bicyclo[2.2.1] heptyl. Examples of such radicals include cyclopropyl, cyclobutyl, cyclopentyl and cyclohexyl. Preferred cycloalkyl groups are cyclopentyl, cyclohexyl, and cycloheptyl. The cycloalkyl groups herein are unsubstituted or, as specified, substituted in one or more substitutable positions with various groups. For example, such cycloalkyl groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆ alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

By “aryl” is meant an aromatic carbocyclic group having a single ring (e.g., phenyl) or multiple condensed rings in which at least one is aromatic, (e.g., 1,2,3,4-tetrahydronaphthyl, naphthyl), which is optionally mono-, di-, or trisubstituted. Preferred aryl groups of the present invention are phenyl, 1-naphthyl, 2-naphthyl, indanyl, indenyl, dihydronaphthyl, fluorenyl, tetralinyl or 6,7,8,9-tetrahydro-5H-benzo[a]cycloheptenyl. The aryl groups herein are unsubstituted or, as specified, substituted in one or more substitutable positions with various groups. For example, such aryl groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

By “heteroaryl” is mean at least one or more aromatic ring systems of 5-, 6-, or 7-membered rings which includes fused ring systems of 9-11 atoms containing at least one and up to four heteroatoms selected from nitrogen, oxygen, or sulfur. Preferred heteroaryl groups of the present invention include pyridinyl, pyrimidinyl, quinolinyl, benzothienyl, indolyl, indolinyl, pryidazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, triazolyl, tetrazolyl, isothiazolyl, naphthyridinyl, isochromanyl, chromanyl, tetrahydroisoquinolinyl, isoindolinyl, isobenzotetrahydrofuranyl, isobenzotetrahydrothienyl, isobenzothienyl, benzoxazolyl, pyridopyridinyl, benzotetrahydrofuranyl, benzotetrahydrothienyl, purinyl, benzodioxolyl, triazinyl, pteridinyl, benzothiazolyl, imidazopyridinyl, imidazothiazolyl, dihydrobenzisoxazinyl, benzisoxazinyl, benzoxazinyl, dihydrobenzisothiazinyl, benzopyranyl, benzothiopyranyl, chromonyl, chromanonyl, pyridinyl-N-oxide, tetrahydroquinolinyl, dihydroquinolinyl, dihydroquinolinonyl, dihydroisoquinolinonyl, dihydrocoumarinyl, dihydroisocoumarinyl, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, bennimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. The heteroaryl groups herein are unsubstituted or, as specified, substituted in one or more substitutable positions with various groups. For example, such heteroaryl groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆alkenyl, C₂-C₆alkynyl, C₁-C₆ haloalkyl, C₁-C₆ haloalkoxy, amino(C₁-C₆)alkyl, mono(C₁-C₆)alkylamino(C₁-C₆)alkyl or di(C₁-C₆)alkylamino(C₁-C₆)alkyl.

By “heterocycle”, “heterocycloalkyl” or “heterocyclyl” is meant one or more carbocyclic ring systems of 4-, 5-, 6-, or 7-membered rings which includes fused ring systems of 9-11 atoms containing at least one and up to four heteroatoms selected from nitrogen, oxygen, or sulfur. Preferred heterocycles of the present invention include morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, tetrahydrothienyl S-oxide, tetrahydrothienyl S,S-dioxide and homothiomorpholinyl S-oxide. The heterocycle groups herein are unsubstituted or, as specified, substituted in one or more substitutable positions with various groups. For example, such heterocycle groups may be optionally substituted with, for example, C₁-C₆ alkyl, C₁-C₆ alkoxy, halogen, hydroxy, cyano, nitro, amino, mono(C₁-C₆)alkylamino, di(C₁-C₆)alkylamino, C₂-C₆ alkenyl, C₂-C₆ alkynyl, C₁-C₆ haloalkyl, C₁-C₆ haloalkoxy, amino(C₁-C₆)alkyl, mono(C₁-C₆)alkylamino(C₁-C₆)alkyl, di(C₁-C₆)alkylamino(C₁-C₆)alkyl or ═O.

Structures were named using Name Pro IUPAC Naming Software, version 5.09, available from Advanced Chemical Development, Inc., 90 Adelaide Street West, Toronto, Ontario, M5H 3V9, Canada or using ChemDraw v. 6.02, ChemDraw v. 8.03, or ChemDraw v. 9.01, all of which are available from Cambridgesoft at 100 Cambridge Park Drive, Cambridge, Mass. 02140 (www.cambridgesoft.com).

The compounds of this invention may contain one or more asymmetric carbon atoms, so that the compounds can exist in different stereoisomeric forms. These compounds can be, for example, racemates, chiral non-racemic or diastereomers. In these situations, the single enantiomers, i.e., optically active forms, can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

Non-toxic pharmaceutically acceptable salts include, but are not limited to salts of inorganic acids such as hydrochloric, sulfuric, phosphoric, diphosphoric, hydrobromic, and nitric or salts of organic acids such as formic, citric, malic, maleic, fumaric, tartaric, succinic, acetic, lactic, methanesulfonic, p-toluenesulfonic, 2-hydroxyethylsulfonic, salicylic and stearic. Similarly, pharmaceutically acceptable cations include, but are not limited to sodium, potassium, calcium, aluminum, lithium and ammonium. Those skilled in the art will recognize a wide variety of non-toxic pharmaceutically acceptable addition salts. The invention also encompasses prodrugs of the compounds of Formula I.

The invention also encompasses the acylated prodrugs of the compounds of Formula I. Those skilled in the art will recognize various synthetic methodologies, which may be employed to prepare non-toxic pharmaceutically acceptable addition salts and acylated prodrugs of the compounds encompassed by Formula I.

The term “acid prodrug group” denotes a moiety that is converted in vivo into an active carboxylic acid compound of formula I. Such prodrug groups are generally known in the art and include ester forming groups, to form an ester prodrug, such as benzyloxy, di(C₁-C₆)alkylaminoethyloxy, acetoxymethyl, pivaloyloxymethyl, phthalidoyl, ethoxycarbonyloxyethyl, 5-methyl-2-oxo-1,3-dioxol-4-yl methyl, and (C₁-C₆)alkoxy optionally substituted by N-morpholino and amide-forming groups such as di(C₁-C₆)alkylamino. Preferred prodrug groups include C₁-C₆ alkoxy forming an ester, and O⁻M⁺ where M⁺ represents a cation to form a salt of the acid. Preferred cations include sodium, potassium, and ammonium. Other cations include magnesium and calcium. Further preferred prodrug groups include O⁼M⁺⁺ where M⁺⁺ is a divalent cation such as magnesium or calcium.

When the compounds described herein contain olefinic double bonds or other centers of geometric asymmetry, and unless otherwise specified, it is intended that the compounds include the cis, trans, Z- and E-configurations. Likewise, all tautomeric forms are also intended to be included.

The invention also encompasses the prodrugs of the compounds of Formula I. Those skilled in the art will recognize various synthetic methodologies that may be employed to prepare non-toxic pharmaceutically acceptable prodrugs of the compounds encompassed by Formula I. Those skilled in the art will recognize a wide variety of non-toxic pharmaceutically acceptable solvates, such as water, ethanol, mineral oil, vegetable oil, and dimethylsulfoxide.

The compounds of general Formula I may be administered orally, topically, parenterally, by inhalation or spray or rectally in dosage unit formulations containing conventional non-toxic pharmaceutically acceptable carriers, adjuvants and vehicles. The term parenteral as used herein includes percutaneous, subcutaneous, intravascular (e.g., intravenous), intramuscular, or intrathecal injection or infusion techniques and the like. In addition, there is provided a pharmaceutical formulation comprising a compound of general Formula I and a pharmaceutically acceptable carrier. One or more compounds of general Formula I may be present in association with one or more non-toxic pharmaceutically acceptable carriers and/or diluents and/or adjuvants, and if desired other active ingredients. The pharmaceutical compositions containing compounds of general Formula I may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide pharmaceutically elegant and palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules, wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Formulations for oral use may also be presented as lozenges.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffm or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono-or diglycerides. In addition, fatty acids such as oleic acid find use in the preparation of injectables.

The compounds of general Formula I may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds of general Formula I may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

For disorders of the eye or other external tissues, e.g., mouth and skin, the formulations are preferably applied as a topical gel, spray, ointment or cream, or as a suppository, containing the active ingredients in a total amount of, for example, 0.075 to 30% w/w, preferably 0.2 to 20% w/w and most preferably 0.4 to 15% w/w. When formulated in an ointment, the active ingredients may be employed with either paraffinic or a water-miscible ointment base.

Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a transdermal device. Preferably topical administration will be accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent may also function as the membrane. The transdermal patch may include the compound in a suitable solvent system with an adhesive system, such as an acrylic emulsion, and a polyester patch. The oily phase of the emulsions of this invention may be constituted from known ingredients in a known manner. While the phase may comprise merely an emulsifier, it may comprise a mixture of at least one emulsifier with a fat or oil or with both a fat and an oil. Preferably, a hydrophilic emulsifier is included together with a lipophilic emulsifier, which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base, which forms the oily, dispersed phase of the cream formulations. Emulsifiers and emulsion stabilizers suitable for use in the formulation of the invention include Tween 60, Span 80, cetostearyl alcohol, myristyl alcohol, glyceryl monostearate, and sodium lauryl sulfate, among others. The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffm or other mineral oils can be used.

Formulations suitable for topical administration to the eye also include eye drops wherein the active ingredients are dissolved or suspended in suitable carrier, especially an aqueous solvent for the active ingredients. The anti-inflammatory active ingredients are preferably present in such formulations in a concentration of 0.5 to 20%, advantageously 0.5 to 10% and particularly about 1.5% w/w. For therapeutic purposes, the active compounds of this combination invention are ordinarily combined with one or more adjuvants appropriate to the indicated route of administration. The compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuiric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose. Formulations for parenteral administration may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

Dosage levels of the order of from about 0.1 mg to about 140 mg per kilogram of body weight per day are useful in the treatment of the above-indicated conditions (about 0.5 mg to about 7 g per patient per day). The amount of active ingredient that may be combined with the carrier materials to produce a single dosage form will vary depending upon the host treated and the particular mode of administration. Dosage unit forms will generally contain between from about 1 mg to about 500 mg of an active ingredient. The daily dose can be administered in one to four doses per day. In the case of skin conditions, it may be preferable to apply a topical preparation of compounds of this invention to the affected area two to four times a day.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

For administration to non-human animals, the composition may also be added to the animal feed or drinking water. It may be convenient to formulate the animal feed and drinking water compositions so that the animal takes in a therapeutically appropriate quantity of the composition along with its diet. It may also be convenient to present the composition as a premix for addition to the feed or drinking water.

The disclosures in this document of all articles and references, including patents, are incorporated herein by reference in their entirety.

The invention is illustrated further by the following examples, which are not to be construed as limiting the invention in scope or spirit to the specific procedures described in them.

The starting materials and various intermediates may be obtained from commercial sources, prepared from commercially available compounds, and/or prepared using known synthetic methods.

General Synthetic Procedures

The compounds of the invention can be prepared using methods known in the art of organic synthesis. For example, the compounds of the invention, as well as all intermediates, can be synthesized by known processes using either solution or solid phase techniques, as shown below. Representative procedures for preparing compounds of the invention are outlined in the following schemes.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Second Edition, Wiley, New York, 1991, and references cited therein.

Compounds of formula II to XI can be prepared by various methods known to those skilled in the art. For example, compounds of formula II to XI can be synthesized by known processes using either solution or solid phase techniques, as shown below. 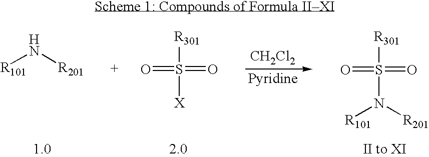
R₁₀₁ and R₂₀₁ are alkyl, alkenyl optionally substituted with a heteroatom and R₃₀₁ is

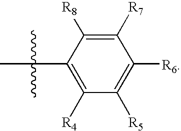

Sulfonylation of secondary cyclic amines 1.0 (R₁ cyclized to R₂) with an appropriate sulfonylhalide 2.0 in a suitable solvent such as dichloromethane, diethylether, acetonitrile, ethyl acetate or acetone, in the presence of a base such as pyridine, diisopropylethylamine or triethylamine, afford compounds of formula II to XI. 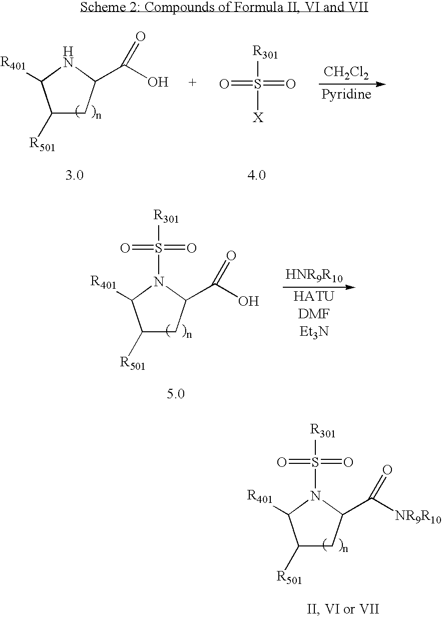
Wherein n is 1 or 2, R₄₀₁ is R₂, R₃, R₂₁, R₂₂, R₁₅ or R₂₄, R₅₀₁ is R₂₃ or R₁, and R₃₀₁ is

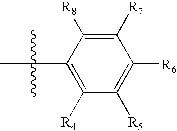

Sulfonylation of amino acids 3.0 with an appropriate sulfonylhalide 4.0 in a suitable solvent such as dichloromethane, diethylether, acetonitrile, ethyl acetate or acetone, in the presence of a base such as pyridine, diisopropylethylamine or triethylamine, afford compounds of formula 5.0. Compounds 5.0 are further functionalized by treatment with a primary (R₆═H) or secondary amine in the presence of a suitable solvent such as dimethylformamide, tetrahydrofuran, dichloromethane, acetonitrile or pyridine with a base such as triethylamine, diisopropylethylamine or pyridine, followed by the addition of O-(7-Azabenzotriazol-1-yl)-tetramethyluronium hexafluorophosphate (HATU), (1-(3-(dimethylamino)propyl)-3-ethylcarbodiimide (EDC) and 1-hydroxybenzotriazole (HOBt) or dicyclohexylcarbodiimide (DCC) and HOBT to afford compounds of formula II, VI or VII. 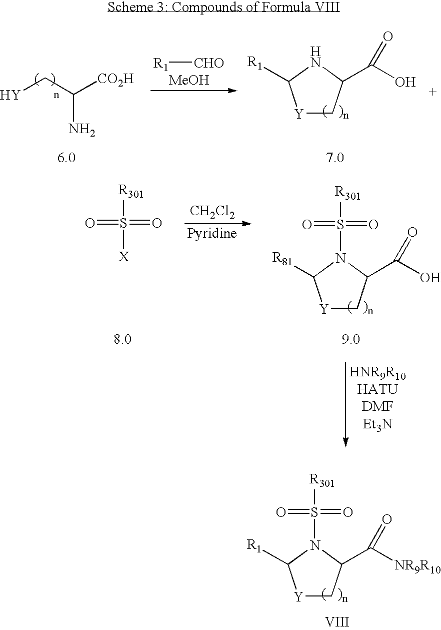
Wherein n is 1 and R₃₀₁ is

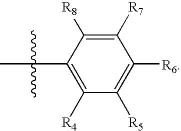

Refluxing primary amines 6.0 with appropriate aldehydes in a suitable solvent such as methanol yields aminoacids 7.0. Subsequent sulfonylation of aminoacids 7.0 with an appropriate sulfonylhalide 8.0 in a suitable solvent such as dichloromethane, diethylether, acetonitrile, ethyl acetate or acetone, in the presence of a base such as pyridine, diisopropylethylamine or triethylamine afford compounds of formula 9.0. Compounds 9.0 are further functionalized by treatment with a primary (R₉═H) or secondary amine in the presence of a suitable solvent such as dimethylformamide, tetrahydrofuran, dichloromethane, acetonitrile or pyridine with a base such as triethylamine, diisopropylethylamine or pyridine, followed by the addition of O-(7-Azabenzotriazol-1-yl)-tetramethyluronium hexafluorophosphate (HATU), (1-(3-(dimethylamino)propyl)-3-ethylcarbodiimide (EDC) and 1-hydroxybenzotriazole (HOBt) or dicyclohexylcarbodiimide (DCC) and HOBT to afford compounds of formula VIII.

Certain compounds of this invention are prepared from other compounds of this invention via-known reactions and functional group transformations. Examples of such transformations are ester hydrolysis, amide formation, and reductive alkylation; with examples of these are described in the preparations below. Starting materials are obtained from commercial sources or prepared by known methods as described in the examples below.

Compounds included in this invention are exemplified by the following examples, which should not be construed as limiting the scope of this disclosure. Analogous structures and alternative synthetic routes within the scope of the invention will be apparent to those skilled in the art.

In the following examples, MH⁺ refers to the mass as determined by LC/MS carried out on a ThermoHypersil-Keystone BDS Hypersil C18 column (50 mm×3 mm, 5 micron particle size). MNa⁺ is used to identify the product based on its sodium adduct. Elution conditions for LC/MS are as follows: Solvents: A. Water with 0.05% TFA (v/v); B. Acetonitrile with 0.05% TFA (v/v); Flow rate: 3 mL/min

Gradient Method

| Time (min) | % B Conc |
|---|---|
| 0 | 5 |
| 0.25 | 5 |
| 2.75 | 95 |
| 3.5 | 95 |
| 3.6 | 5 |
| 4.0 | STOP |

To isolate compounds of the following examples, a Varian reverse-phase preparative HPLC, was employed utilizing a Phenomenex Aqua C₁₈ column (60 mm×21.2 mm, 5 micron particle size). Elution conditions for the HPLC are as follows: Solvents: A. Water with 0.1% TFA (v/v); B. Acetonitrile with 0.1% TFA (v/v); Flow rate: 25 mL/min

Gradient Method

| Time (min) | % B Conc |
|---|---|
| 0 | 5 |
| 0.75 | 5 |
| 9.5 | 100 |
| 10.5 | 100 |
| 11.5 | 5 |
| 12.0 | STOP |

### EXAMPLE 1

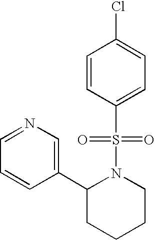

A mixture of commercially available DL-anabasine (64.9 mg; 0.4 mmol) and pyridine (252 ul; 3.2 mmol) was stirred in CH₂Cl₂ at 0° C. for 1 h. 4-chlorobenzenesulfonylchloride (126.6 mg; 0.6 mmol) was added and subsequently stirred at 10° C. for 18 h. The crude reaction mixture was then purified on a Varian reverse-phase preparative HPLC to afford the product example 1

### EXAMPLE 2

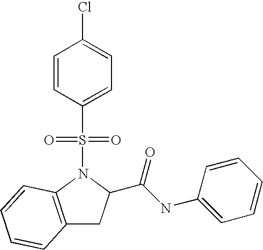 Step 1

A mixture of DL-indoline-2-carboxylic acid (50 mg; 0.31 mmol) and pyridine (97 ul; 1.24 mmol) was stirred in CH₂Cl₂ at 0° C. for 1 h. 4-chlorobenzenesulfonylchloride (97 mg; 0.46 mmol) was added and subsequently stirred at 10° C. for 24 h. The crude reaction mixture was then purified on a Varian reverse-phase preparative HPLC to afford the desired product.

Step 2

A portion of the product formed in Step 1 (0.06 mmol) was dissolved in DMF and treated with aniline (5.6 ul; 0.06 mmol) and triethylamine (33 ul; 0.24 mmol). Subsequent addition of HATU (23 mg; 0.06 mmol) facilitated the amide bond formation. The resulting crude reaction mixture was then purified on a Varian reverse-phase preparative HPLC to afford the product example 2.

### EXAMPLE 3

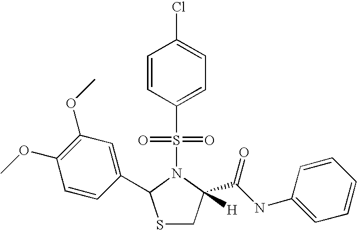 Step 1

A mixture of 3,4-dimethoxybenzaldehyde (1.66 g; 10 mmol) and L-cysteine (1.21 g; 10 mmol) was heated to reflux for 18 h in methanol. The reaction was then cooled in an ice bath and resulting cyclized product was filtered and washed with cold methanol.

Step 2

A portion of the product from Step 1 (484 mg; 1.8 mmol) and pyridine (711 ul; 9 mmol) was stirred in CH₂Cl₂ at 0° C. for 1 h. 4-chlorobenzenesulfonylchloride (570 mg; 2.7 mmol) was added and subsequently stirred at 0° C. for 24 h. The crude reaction mixture was then purified on a Varian reverse-phase preparative HPLC to afford the desired product.

Step 3

A portion of the product formed in Step 2 (0.033 mmol) was dissolved in DMF and treated with aniline (3.1 ul; 0.033 mmol) and triethylamine (18 ul; 0.13 mmol). Subsequent addition of HATU (12 mg; 0.033 mmol) facilitated the amide bond formation. The resulting crude reaction mixture was then purified on a Varian reverse-phase preparative HPLC to afford the product in Example 3(138).

The following compounds were prepared essentially according to the methods and procedures described above.

| Ex. No. | Name | M + H+ | M + Na+ |
|---|---|---|---|
| 1 | 1-(4-Chloro-benzenesulfonyl)-1,2,3,4,5,6-hexahydro- | 337.0 | — |
| | [2,3′]bipyridinyl | | |
| 2 | 1-(4-Chloro-benzenesulfonyl)-2,3-dihydro-1H-indole- | 412.9 | — |
| | 2-carboxylic acid phenylamide | | |
| 3 | (4S)-N-benzyl-3-[(4-chlorophenyl)sulfonyl]-2-(3,4- | 532.7 | — |
| | dimethoxyphenyl)-1,3-thiazolidine-4-carboxamide | | |
| 4 | (3R)-N-(tert-butyl)-2-[(4- | 413.1 | — |
| | chlorophenyl)sulfonyl]decahydroisoquinoline-3- | | |
| | carboxamide | | |
| 5 | 1′-[(4-chlorophenyl)sulfonyl]spiro[indene-1,4′- | 360.0 | — |
| | piperidine] | | |
| 6 | (2S)-1-[(4-chlorophenyl)sulfonyl]octahydro-1H-indole- | 344.0 | — |
| | 2-carboxylic acid | | |
| 7 | 8-(4-Chloro-benzenesulfonyl)-1-phenyl-1,3,8-triaza- | 406.1 | — |
| | spiro[4.5]decan-4-one | | |
| 8 | [8-(4-Chloro-benzenesulfonyl)-7,7,9,9-tetramethyl-1,4- | 403.9 | — |
| | dioxa-8-aza-spiro[4.5]dec-2-yl]-methanol | | |
| 9 | 1-(4-Chloro-benzenesulfonyl)-decahydro-quinoline | 314.1 | — |
| 10 | (1s,5s)-3-[(4-chlorophenyl)sulfonyl]-3- | 299.9 | — |
| | azabicyclo[3.2.2]nonane | | |
| 11 | 2-(4-Chloro-benzenesulfonyl)-1-methyl-2,3,4,9- | 405.1 | — |
| | tetrahydro-1H-b-carboline-3-carboxylic acid | | |
| 12 | 1-(4-Chloro-benzenesulfonyl)-2,3-dihydro-1H-indole- | 427.0 | — |
| | 2-carboxylic acid benzylamide | | |
| 13 | 1-(4-Chloro-benzenesulfonyl)-6-fluoro-2-methyl- | — | 362.0 |
| | 1,2,3,4-tetrahydro-quinoline | | |
| 14 | 1-(4-Chloro-benzenesulfonyl)-2,3-dihydro-1H-indole- | 393.0 | — |
| | 2-carboxylic acid butylamide | | |
| 15 | 5-(4-Chloro-benzenesulfonyl)-10,11-dihydro-5H- | — | 392.0 |
| | dibenzo[b,f]azepine | | |
| 16 | 5-(4-Chloro-benzenesulfonyl)-5H-dibenzo[b,f]azepine | — | 390.0 |
| 17 | 4-(4-Chloro-benzenesulfonyl)-3-methyl-3,4-dihydro- | 323.8 | — |
| | 2H-benzo[1,4]oxazine | | |
| 18 | 4-(4-Chloro-benzenesulfonyl)-3-methyl-3,4-dihydro- | 337.0 | — |
| | 1H-quinoxalin-2-one | | |
| 19 | 8-(4-Chloro-benzenesulfonyl)-1,4-dioxa-8-aza- | 318.0 | — |
| | spiro[4.5]decane | | |
| 20 | 4-(4-Chloro-benzenesulfonyl)-3-phenyl-3,4-dihydro- | 385.9 | — |
| | 2H-benzo[1,4]oxazine | | |
| 21 | 1-(4-Chloro-benzenesulfonyl)-1,2,3,4-tetrahydro- | — | 330.0 |
| | quinoline | | |
| 22 | 4-(4-Chloro-benzenesulfonyl)-3,4-dihydro-1H- | 323.0 | — |
| | quinoxalin-2-one | | |
| 23 | 2-(4-Chloro-benzenesulfonyl)-1,2,3,4-tetrahydro- | 308.0 | — |
| | isoquinoline | | |
| 24 | 1-(4-Chloro-benzenesulfonyl)-2,3-dihydro-1H-indole- | 337.3 | — |
| | 2-carboxylic acid | | |
| 25 | 1-(4-Chloro-benzenesulfonyl)-5-methoxy-1H-indole-2- | 366.1 | — |
| | carboxylic acid | | |
| 26 | 1-(4-Chloro-benzenesulfonyl)-2,3-dihydro-1H-indole- | 337.1 | — |
| | 2-carboxylic acid amide | | |
| 27 | 1-(4-Chloro-benzenesulfonyl)-2,3-dihydro-1H-indole- | 351.0 | — |
| | 2-carboxylic acid methylamide | | |
| 28 | 1-(4-Chloro-benzenesulfonyl)-2,3-dihydro-1H-indole- | 365.0 | — |
| | 2-carboxylic acid dimethylamide | | |
| 29 | [1-(4-Chloro-benzenesulfonyl)-2,3-dihydro-1H-indol-2- | 391.0 | — |
| | yl]-pyrrolidin-1-yl-methanone | | |
| 30 | 1-(4-Chloro-benzenesulfonyl)-2,3-dihydro-1H-indole- | 441.0 | — |
| | 2-carboxylic acid benzyl-methyl-amide | | |
| 31 | 10-(4-Chloro-benzenesulfonyl)-10H-phenothiazine | — | 396.0 |
| 32 | 1-(4-Chloro-benzenesulfonyl)-6-ethoxy-2,2,4-trimethyl- | 392.1 | — |
| | 1,2-dihydro-quinoline | | |
| 33 | 1-(4-Chloro-benzenesulfonyl)-1H-naphtho[1,2- | 358.9 | — |
| | d]imidazol-7-ol | | |
| 34 | 1-(4-Chloro-benzenesulfonyl)-1,2,3,4-tetrahydro- | 323.0 | — |
| | quinolin-8-ylamine | | |
| 35 | 1-(4-Chloro-benzenesulfonyl)-5-nitro-2,3-dihydro-1H- | 339.0 | — |
| | indole | | |
| 36 | 2-(4-Chloro-benzenesulfonyl)-6,7-dimethoxy-1,2,3,4- | 368.0 | — |
| | tetrahydro-isoquinoline | | |
| 37 | 1-(4-Chloro-benzenesulfonyl)-6-nitro-2,3-dihydro-1H- | 339.0 | — |
| | indole | | |
| 38 | [2-(4-Chloro-benzenesulfonyl)-1,2,3,4-tetrahydro- | 365.1 | — |
| | isoquinolin-1-yl]-acetic acid | | |
| 39 | 1-(4-Chloro-benzenesulfonyl)-2-methylsulfanyl-1H- | 339.0 | — |
| | benzoimidazole | | |
| 40 | 1-(4-Chloro-benzenesulfonyl)-1H-indazole- | 293.0 | — |
| 41 | 10-(4-Chloro-benzenesulfonyl)-1,4,7-trioxa-10-aza- | 350.1 | — |
| | cyclododecane | | |
| 42 | 1-(4-Chloro-benzenesulfonyl)-[1,4]diazepane | 275.0 | — |
| 43 | 1-(4-Chloro-benzenesulfonyl)-azepane | 274.1 | — |
| 44 | 1-(4-Chloro-benzenesulfonyl)-piperidine | 260.0 | — |
| 45 | (2R,6S)-1-[(4-chlorophenyl)sulfonyl]-2,6- | 288.0 | — |
| | dimethylpiperidine | | |
| 46 | 1-(4-Chloro-benzenesulfonyl)-2-ethyl-piperidine | 288.0 | — |
| 47 | 1-(4-Chloro-benzenesulfonyl)-2,6-dimethyl-piperidine | 287.7 | — |
| 48 | 1-(4-Chloro-benzenesulfonyl)-2-methyl-piperidine | 273.8 | — |
| 49 | [1-(4-Chloro-benzenesulfonyl)-piperidin-2-yl]- | 290.0 | — |
| | methanol | | |
| 50 | 2-(4-Chloro-benzenesulfonyl)-6-methoxy-2,3,4,9- | 377.0 | — |
| | tetrahydro-1H-b-carboline | | |
| 51 | 1-(4-Chloro-benzenesulfonyl)-3,5-dimethyl-piperidine | 288.0 | — |
| 52 | 1-(4-Chloro-benzenesulfonyl)-4-methyl-piperidine | 273.8 | — |
| 53 | 2-[1-(4-Chloro-benzenesulfonyl)-piperidin-4-yl]- | 303.8 | — |
| | ethanol | | |
| 54 | 1-(4-Chloro-benzenesulfonyl)-3-methyl-piperidine | 273.8 | — |
| 55 | 1-(4-Chloro-benzenesulfonyl)-piperidin-4-ol | 276.0 | — |
| 56 | 4-Bromo-1-(4-chloro-benzenesulfonyl)-piperidine | 338.9 | — |
| 57 | 1-(4-Chloro-benzenesulfonyl)-piperidine-3-ol | 276.0 | — |
| 58 | [1-(4-Chloro-benzenesulfonyl)-piperidine-3-yl]- | 290.0 | — |
| | methanol | | |
| 59 | [1-(4-Chloro-benzenesulfonyl)-piperidin-4-yl]- | 290.0 | — |
| | methanol | | |
| 60 | 1-(4-Chloro-benzenesulfonyl)-4-propyl-piperidine | 302.0 | — |
| 61 | 1-(4-Chloro-benzenesulfonyl)-piperidine-3-carboxylic | 303.9 | — |
| | acid | | |
| 62 | 1-(4-Chloro-benzenesulfonyl)-piperidine-4-carboxylic | 304.0 | — |
| | acid | | |
| 63 | 1-(4-Chloro-benzenesulfonyl)-piperidine-3-carboxylic | 359.0 | — |
| | acid diethylamide | | |
| 64 | 1-(4-Chloro-benzenesulfonyl)-piperidine-4-carboxylic | 332.0 | — |
| | acid ethyl ester | | |
| 65 | 1-(4-Chloro-benzenesulfonyl)-piperidine-3-carboxylic | 332.0 | — |
| | acid ethyl ester | | |
| 66 | {2-[1-(4-Chloro-benzenesulfonyl)-piperidin-4-yl]- | — | 425.1 |
| | ethyl}-carbamic acid tert-butyl ester | | |
| 67 | 1-(4-Chloro-benzenesulfonyl)-4-phenyl-piperidine | 336.0 | — |
| 68 | 4-Benzyl-1-(4-chloro-benzenesulfonyl)-piperidine | 350.0 | — |
| 69 | 1′-(4-Chloro-benzenesulfonyl)-[1,4′]bipiperidinyl | 343.1 | — |
| 70 | 2-(4-{3-[1-(4-Chloro-benzenesulfonyl)-piperidine-4-yl]- | 429.2 | — |
| | propyl}-piperidin-1-yl)-ethanol | | |
| 71 | [1-(4-Chloro-benzenesulfonyl)-piperidin-4-yl]- | — | 464.0 |
| | diphenyl-methanol | | |
| 72 | 1-[1-(4-Chloro-benzenesulfonyl)-piperidin-4-yl]-1,3- | 392.0 | — |
| | dihydro-benzoimidazol-2-one | | |
| 73 | 1-(4-Chloro-benzenesulfonyl)-4-oxo-piperidine-3- | 346.0 | — |
| | carboxylic acid ethyl ester | | |
| 74 | 1-(4-Chloro-benzenesulfonyl)-3-methyl-3-phenyl- | 349.8 | — |
| | piperidine | | |
| 75 | 1-(4-Chloro-benzenesulfonyl)-4-(4-chloro-phenyl)- | 386.0 | — |
| | piperidin-4-ol | | |
| 76 | 1-(4-Chloro-benzenesulfonyl)-4-phenyl-piperidine-4- | 361.0 | — |
| | carbonitrile | | |
| 77 | 1-(4-Chloro-benzenesulfonyl)-4-phenyl-piperidin-4-ol | 352.0 | — |
| 78 | 1-[1-(4-Chloro-benzenesulfonyl)-4-phenyl-piperidin-4- | 378.0 | — |
| | yl]-ethanone | | |
| 79 | [1-(4-Chloro-benzenesulfonyl)-3-oxo-piperazin-2-yl]- | 361.0 | — |
| | acetic acid ethyl ester | | |
| 80 | (3S)-1-[(4-chlorophenyl)sulfonyl]-3-methylpiperazine | 275.0 | — |
| 81 | (3R)-1-[(4-chlorophenyl)sulfonyl]-3-methylpiperazine | 275.0 | — |
| 82 | 1-(4-Chloro-benzenesulfonyl)-4-ethyl-piperazine | 289.1 | — |
| 83 | 2-[4-(4-Chloro-benzenesulfonyl)-piperazin-1-yl]- | 305.0 | — |
| | ethanol | | |
| 84 | 2-{2-[4-(4-Chloro-benzenesulfonyl)-piperazin-1-yl]- | 349.0 | — |
| | ethoxy}-ethanol | | |
| 85 | 4-(4-Chloro-benzenesulfonyl)-piperazine-1-carboxylic | 333.0 | — |
| | acid ethyl ester | | |
| 86 | 4-(4-Chloro-benzenesulfonyl)-piperazine-1-carboxylic | — | 383.0 |
| | acid tert-butyl ester | | |
| 87 | 2-[4-(4-Chloro-benzenesulfonyl)-piperazin-1-yl]-1- | 372.1 | — |
| | pyrrolidin-1-yl-ethanone | | |
| 88 | [4-(4-Chloro-benzenesulfonyl)-piperazin-1-yl]-furan-2- | 355.0 | — |
| | yl-methanone | | |
| 89 | 4-(4-Chloro-benzenesulfonyl)-piperazine-1-carboxylic | — | 417.0 |
| | acid benzyl ester | | |
| 90 | 1-Benzyl-4-(4-chloro-benzenesulfonyl)-piperazine | 351.0 | — |
| 91 | 4-(4-Chloro-benzenesulfonyl)-2-methyl-1-phenyl- | 351.0 | — |
| | piperazine | | |
| 92 | 1-(4-Chloro-benzenesulfonyl)-4-(4-Chloro-benzyl)- | 385.0 | — |
| | piperazine | | |
| 93 | 1-(4-Chloro-benzenesulfonyl)-4-o-tolyl-piperazine | 351.0 | — |
| 94 | 4-(4-Chloro-benzenesulfonyl)-2-methyl-1-p-tolyl- | 365.1 | — |
| | piperazine | | |
| 95 | 4-(4-Chloro-benzenesulfonyl)-1-(4-methoxy-phenyl)-2- | 381.0 | — |
| | methyl-piperazine | | |
| 96 | 1-(4-Chloro-benzenesulfonyl)-4-(2-methoxy-phenyl)- | 367.0 | — |
| | piperazine | | |
| 97 | 1-(4-Chloro-benzenesulfonyl)-4-(4-fluoro-phenyl)- | 355.0 | — |
| | piperazine | | |
| 98 | 1-(4-Chloro-benzenesulfonyl)-4-(3-Chloro-phenyl)- | 371.0 | — |
| | piperazine | | |
| 99 | 1-(4-Chloro-benzenesulfonyl)-4-(3,4-dichloro-phenyl)- | 406.0 | — |
| | piperazine | | |
| 100 | 1-(4-Chloro-benzenesulfonyl)-4-(3,5-dichloro-phenyl)- | 406.0 | — |
| | piperazine | | |
| 101 | 1-(4-Chloro-benzenesulfonyl)-4-phenethyl-piperazine | 365.1 | — |
| 102 | 1-Benzhydryl-4-(4-chloro-benzenesulfonyl)-piperazine | 427.1 | — |
| 103 | 1-(4-Chloro-benzenesulfonyl)-4-(1-phenyl-ethyl)- | 365.1 | — |
| | piperazine | | |
| 104 | 1-(4-Chloro-benzenesulfonyl)-4-(3-trifluoromethyl- | 405.1 | — |
| | phenyl)-piperazine | | |
| 105 | 1-(4-Chloro-benzenesulfonyl)-4-(4-chloro-phenyl)- | 371.0 | — |
| | piperazine | | |
| 106 | 2-[4-(4-Chloro-benzenesulfonyl)-piperazine-1-yl]- | 362.0 | — |
| | benzonitrile | | |
| 107 | 1-(4-Chloro-benzenesulfonyl)-4-(2,3-dimethyl-phenyl)- | 365.1 | — |
| | piperazine | | |
| 108 | 1-(4-Chloro-benzenesulfonyl)-4-p-tolyl-piperazine | 351.0 | — |
| 109 | 4-(4-Chloro-benzenesulfonyl)-2-methyl-1-m-tolyl- | 365.1 | — |
| | piperazine | | |
| 110 | 1-Benzo[1,3]dioxol-5-ylmethyl-4-(4-chloro- | 395.0 | — |
| | benzenesulfonyl)-piperazine | | |
| 111 | 4-[4-(4-Chloro-benzenesulfonyl)-piperazin-1-yl]-2- | 456.0 | — |
| | trifluoromethyl-quinoline | | |
| 112 | 2-[4-(4-Chloro-benzenesulfonyl)-piperazin-1-yl]- | 339.0 | — |
| | pyrimidine | | |
| 113 | 1-(4-Chloro-benzenesulfonyl)-4-pyridin-4-yl-piperazine | 338.0 | — |
| 114 | 1-(4-Chloro-benzenesulfonyl)-4-pyridin-2-yl-piperazine | 338.0 | — |
| 115 | 1-(4-Chloro-benzenesulfonyl)-4-(5-trifluoromethyl- | 406.0 | — |
| | pyridin-2-yl)-piperazine | | |
| 116 | 4-(4-Chloro-benzenesulfonyl)-2,6-dimethyl-morpholine | 290.0 | — |
| 117 | 4-(4-Chloro-benzenesulfonyl)-morpholine | 262.0 | — |
| 118 | 4-(4-Chloro-benzenesulfonyl)-thiomorpholine | 278.0 | — |
| 119 | 1-(4-Chloro-benzenesulfonyl)-pyrrolidin-3-ol | 262.0 | — |
| 120 | (3S)-1-[(4-chlorophenyl)sulfonyl]pyrrolidin-3-ol | 262.0 | — |
| 121 | tert-butyl{(3S)-1-[(4-chlorophenyl)sulfonyl]pyrrolidin- | — | 383.0 |
| | 3-yl}carbamate | | |
| 122 | tert-butyl{(3R)-1-[(4-chlorophenyl)sulfonyl]pyrrolidin- | — | 383.0 |
| | 3-yl}carbamate | | |
| 123 | [1-(4-Chloro-benzenesulfonyl)-pyrrolidin-3-yl]- | — | 383.0 |
| | carbamic acid tert-butyl ester | | |
| 124 | 1-(4-Chloro-benzenesulfonyl)-2,5-dimethyl-pyrrolidine | 274.0 | — |
| 125 | (2S)-1-[(4-chlorophenyl)sulfonyl]-2-(pyrrolidin-1- | 329.1 | — |
| | ylmethyl)pyrrolidine | | |
| 126 | benzyl 1-[(4-chlorophenyl)sulfonyl]-D-prolinate | 380.0 | — |
| 127 | benzyl 1-[(4-chlorophenyl)sulfonyl]-L-prolinate | 380.0 | — |
| 128 | N-({(2R)-1-[(4-chlorophenyl)sulfonyl]pyrrolidin-2- | 351.1 | — |
| | yl}methyl)aniline | | |
| 129 | {(2S)-1-[(4-chlorophenyl)sulfonyl]pyrrolidin-2- | — | 450.0 |
| | yl}(diphenyl)methanol | | |
| 130 | (2R,5R)-1-[(4-chlorophenyl)sulfonyl]-2,5- | 274.0 | — |
| | dimethylpyrrolidine | | |
| 131 | 1-(4-Chloro-benzenesulfonyl)-5,5-diphenyl- | 427.0 | — |
| | imidazolidine-2,4-dione | | |
| 132 | 1-(4-Chloro-benzenesulfonyl)-5-phenyl-5-p-tolyl- | 441.0 | — |
| | imidazolidine-2,4-dione | | |
| 133 | 1-(4-Chloro-benzenesulfonyl)-5-(3-hydroxy-phenyl)-5- | 443.0 | — |
| | phenyl-imidazolidine-2,4-dione | | |
| 134 | 3-(4-Chloro-benzenesulfonyl)-4,4-dimethyl-oxazolidine | — | 298.0 |
| 135 | 3-(4-Chloro-benzenesulfonyl)-thiazolidine | 264.0 | — |
| 136 | (4S)-3-[(4-chlorophenyl)sulfonyl]-2-(3,4- | 518.8 | — |
| | dimethoxyphenyl)-N-phenyl-1,3-thiazolidine-4- | | |
| | carboxamide | | |
| 137 | (4R)-N-(sec-butyl)-3-[(4-chlorophenyl)sulfonyl]-2-(3,4- | 498.8 | — |
| | dimethoxyphenyl)-1,3-thiazolidine-4-carboxamide | | |
| 138 | 1-(4-Chloro-benzenesulfonyl)-1,3,4,6,7,8-hexahydro- | 314.0 | — |
| | 2H-pyrimido[1,2-a]pyrimidine | | |
| 139 | (4S)-N-butyl-3-[(4-chlorophenyl)sulfonyl]-2-(3,4- | 498.8 | — |
| | dimethoxyphenyl)-1,3-thiazolidine-4-carboxamide | | |
| 140 | (4R)-N-butyl-3-[(4-chlorophenyl)sulfonyl]-2-(3,4- | 498.7 | — |
| | dimethoxyphenyl)-1,3-thiazolidine-4-carboxamide | | |
| 141 | (4R)-3-[(4-chlorophenyl)sulfonyl]-N-cyclohexyl-2-(3,4- | 524.8 | — |
| | dimethoxyphenyl)-1,3-thiazolidine-4-carboxamide | | |
| 142 | (4S)-3-[(4-chlorophenyl)sulfonyl]-2-(3,4- | 518.7 | — |
| | dimethoxyphenyl)-N-phenyl-1,3-thiazolidine-4- | | |
| | carboxamide | | |
| 143 | (4S)-N-benzyl-3-[(4-chlorophenyl)sulfonyl]-2-(3,4- | 532.7 | — |
| | dimethoxyphenyl)-1,3-thiazolidine-4-carboxamide | | |
| 144 | (4S)-3-[(4-chlorophenyl)sulfonyl]-N-cyclohexyl-2-(3,4- | 524.8 | — |
| | dimethoxyphenyl)-1,3-thiazolidine-4-carboxamide | | |
| 145 | 2-(4-Chloro-benzenesulfonyl)-4-iodo-2H-pyrazole-3- | 412.7 | — |
| | carboxylic acid | | |
| 146 | 4-Bromo-1-(4-chloro-benzenesulfonyl)-3-methyl-1H- | — | 358.9 |
| | pyrazole | | |
| 147 | 4-Chloro-2-[1-(4-chloro-benzenesulfonyl)-1H-pyrazol- | 369.9 | — |
| | 3-yl]-phenol | | |
| 148 | 1-(4-Chloro-benzenesulfonyl)-1,2,3,6-tetrahydro- | 258.0 | — |
| | pyridine | | |
| 149 | 1-(4-Chloro-benzenesulfonyl)-2,5-dimethyl-2,5- | 272.0 | — |
| | dihydro-1H-pyrrole | | |
| 150 | 1-(4-Chloro-benzenesulfonyl)-2,5-dihydro-1H-pyrrole | 244.0 | — |
| 151 | 1-(4-Chloro-benzenesulfonyl)-azetidine | 232.0 | — |
| 152 | 1-(4-Chloro-benzenesulfonyl)-2-phenyl-aziridine | 293.7 | — |

Notch Signaling Assay for Selective Inhibitors of Gamma Secretase.

A convergence of evidence indicates that the gamma secretase complex, comprised of the presenilin subunits, mediates the intra-membrane cleavage of Amyloid precursor protein (APP), and the Notch family of proteins (De Strooper, B., P. Saftig, K. Craessaerts, H. Vanderstichele, G. Guhde, W. Annaert, K. Von Figura and F. Van Leuven (1998). “Deficiency of presenilin-1 inhibits the normal cleavage of amyloid precursor protein.” Nature 391(6665): 387-90; De Strooper, B., W. Annaert, P. Cupers, P. Saftig, K. Craessaerts, J. S. Mumm, E. H. Schroeter, V. Schrijvers, M. S. Wolfe, W. J. Ray et al. (1999). “A presenilin-1-dependent gamma-secretase-like protease mediates release of Notch intracellular ” Nature 398(6727): 518-22; Mumm, J. S., E. H. Schroeter, M. T. Saxena, A. Griesemer, X. Tian, D. J. Pan, W. J. Ray and R. Kopan (2000). “A ligand-induced extracellular cleavage regulates gamma-secretase-like proteolytic activation of Notch1.” Mol Cell 5(2): 197-206; Zhang, Z., P. Nadeau, W. Song, D. Donoviel, M. Yuan, A. Bernstein and B. A. Yankner (2000). “Presenilins are required for gamma-secretase cleavage of beta-APP and transmembrane cleavage of Notch-1” Nat Cell Biol 2(7): 463-5). Cleavage of APP by gamma secretase leads to beta-amyloid synthesis. Cleavage of Notch1 by gamma secretase results in release of the Notch intracellular domain (NICD), which translocates to the nucleus and activates gene expression (Jarriault, S., C. Brou, F. Logeat, E. H. Schroeter, R. Kopan and A. Israel (1995). “Signalling downstream of activated mammalian Notch.” Nature 377(6547): 355-8; Kopan, R., E. H. Schroeter, H. Weintraub and J. S. Nye (1996). “Signal transduction by activated Notch: importance of proteolytic processing and its regulation by the extracellular domain.” Proc Natl Acad Sci USA 93(4): 1683-8; Schroeter, E. H., J. A. Kisslinger and R. Kopan (1998). “Notch-1 signalling requires ligand-induced proteolytic release of intracellular domain.” Nature 393(6683): 382-6). In particular, Notch signaling activates transcription of the mammalian homolog of the Drosophila transcription factor hairy-enhancer of split (Hes). Transcriptional activation of Hesl is mediated by de-repression of CBF1/RBPJk upon binding by NICD in the nucleus. These facts have been exploited to develop a reporter gene assay for Notch Signaling Hsieh, J. J., T. Henkel, P. Salmon, E. Robey, M. G. Peterson and S. D. Hayward (1996). “Truncated mammalian Notch1 activates CBF1/RBPJk-repressed genes by a mechanism resembling that of Epstein-Barr virus EBNA2. ” Mol Cell Biol 16(3): 952-9; Lu, F. M. and S. E. Lux (1996). “Constitutively active human Notch1 binds to the transcription factor CBF1 and stimulates transcription through a promoter containing a CBF1-responsive element.” Proc Natl Acad Sci USA 93(11): 5663-7).

Gamma secretase inhibitors have been observed to block NICD formation, and inhibit Notch signaling (De Strooper, B., W. Annaert, P. Cupers, P. Saftig, K. Craessaerts, J. S. Mumm, E. H. Schroeter, V. Schrijvers, M. S. Wolfe, W. J. Ray et al. (1999). “A presenilin-1-dependent gamma-secretase-like protease mediates release of Notch intracellular domain.” Nature 398(6727): 518-22). Due to the importance of Notch signaling in cell fate determination, and tissue differentiation during both development and in the adult, inhibition of Notch signaling by gamma secretase inhibitors is postulated to be a limiting factor in their therapeutic utility. In order to identify selective gamma secretase inhibitors, we have employed a reporter gene based Notch signaling assay using a constitutively active rat Notch1 construct (ZEDN1) provided by Dr Gerry Weinmaster, who is at the University of California at Los Angeles (UCLA) as described in Shawber, C., D. Nofziger, J. J. Hsieh, C. Lindsell, O. Bogler, D. Hayward and G. Weinmaster (1996). “Notch signaling inhibits muscle cell differentiation through a CBF1-independent pathway.” Development 122(12): 3765-73 in combination with the CBF1 repressible Luciferase reporter gene 4xwtCBF1Luc (Hsieh, J. J., T. Henkel, P. Salmon, E. Robey, M. G. Peterson and S. D. Hayward (1996). “Truncated mammalian Notch1 activates CBF1/RBPJk-repressed genes by a mechanism resembling that of Epstein-Barr virus EBNA2. ” Mol Cell Biol 16(3): 952-9).

When 4xwtCBF1 Luciferase is co-transfected with NotchδE (ZEDN1), gamma-secretase cleavage of NotchδE releases the Notch intracellular domain (NICD), which translocates to the nucleus and de-represses CBF1 mediated transcriptional repression, leading to transcription of the Luciferase reporter gene. Luciferase activity is easily assayed in cell extracts using commercially available kits. The activity of the reporter gene is directly correlated with gamma secretase cleavage of NotchδE, and as such, a reduction in Luciferase activity provides a convenient measure of inhibition of gamma secretase cleavage of NotchδE. A comparison of the IC₅₀ values of compounds for inhibition of Notch signaling versus inhibition of beta-amyloid production in 293sw cells is employed to guide in the selection of compounds that have the desired property of potent inhibition of beta-amyloid synthesis with minimal inhibition of Notch Signaling.

Compounds 45, 46, and 47, exhibit an IC₅₀ within the range of from about 100 to 1000 nM; compounds 1, 2, 9, 12, 13, 43, 48, 51, 54, 124, and 149, exhibit an IC₅₀ within the range of from about 1000 to 10,000 nM; compounds 56, 65, 74, 116, 138, and 139, exhibit an IC₅₀ of greater than about 10,000 nM.

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims. To particularly point out and distinctly claim the subject matter regarded as invention, the following claims conclude this specification.

What is claimed is:

1. A compounds of the formula 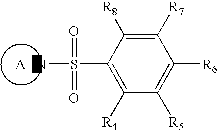
or pharmaceutically acceptable salts thereof, wherein
A-ring is selected from 1,2,3,4-tetrahydroisoquinolinyl, 1,2,3,4-tetrahydroquinolinyl, 1,2,3,4-tetrahydroquinoxalinyl, 1,2-dihydroquinolinyl, 1,3,4,6,7,8-Hexahydro-2H-pyrimido[1,2-a]pyrimidinyl, 1,3,8-triazaspiro[4.5]decan-4-onyl, 1,4,7-trioxa-10-azacyclododecanyl, 1,4-diazepanyl, 1H-naphtho[1,2-d]imidazolyl, 3,4-dihydro-2H-1,4-benzoxazinyl, azepanyl, decahydroisoquinolinyl, decahydroquinolinyl, indolinyl, octahydro-1H-indolyl, 3-azabicyclo[3.2.2]nonanyl, 1H-benzimidazolyl, indazolyl, indolyl, spiro[indene-1,4′-piperidinyl], 5H-dibenzo[b,f]azepinyl, 2-Hydroxymethyl-1,4-dioxa-8-azaspiro[4.5]decanyl, 10H-phenothiazinyl, 1,2,4,5-tetrahydrospiro[2-benzazepine-3,1′-cyclohexanyl], 2,3 ,4,9-tetrahydro-1H-β-carbolinyl, and 10,11-dihydro-5H-dibenzo[b,f]azepinyl, wherein each of the above groups is optionally substituted with 1, 2, 3 or 4 groups that are independently OH, H, CN, oxo, halo, C₁-C₆ alkoxy, C₁-C₆ alkyl, —C(O)NR₉R₁₀, —C(O)N(R₉)—C₁-C₆ alkyl-R₂₆, —S—C₁-C₆ alkyl, —C(O)R₂₈, C₀-C₆ alkyl-R₂₆, C₀-C₆ alkyl-C(O)OR₁₁, C₀-C₆ alkyl-NR₉C(O)OR₁₁, NH₂, mono- or di-(C₁-C₆ alkyl)amino, C₀-C₆ alkyl-C(O)OR₁₁, CF₃, —OCF₃, or NO₂; or
the A-ring is a group having the formula 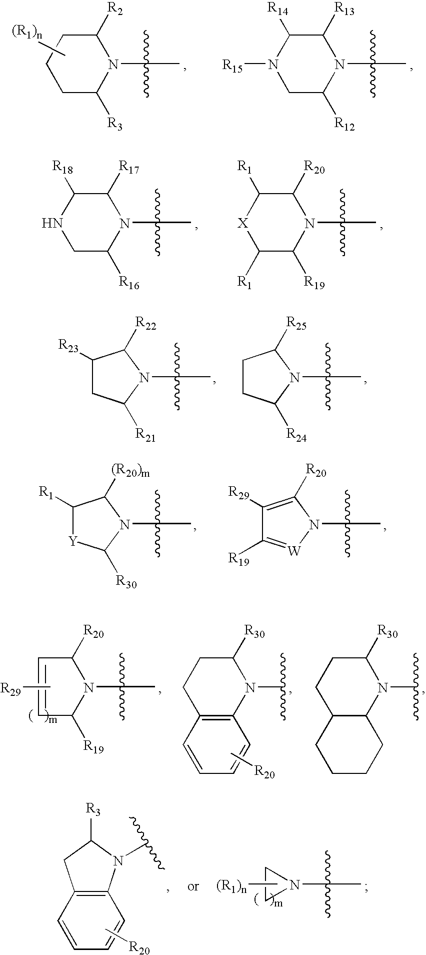
wherein
W is CR₉ or nitrogen;
X is sulfur, SO₂, SO, or oxygen;
Y is sulfur, SO₂, SO, oxygen or NR₉;
m is 1 or 2;
n is 0 or an integer from 1 to 8;
R₁ at each occurrence is independently OH, H, CN, oxo, halo, C₁-C₆ alkyl, C₁-C₆ alkoxy, —C(O)R₁₁, —C(O)NR₉R₁₀, —C(O)OR₁₁, C₀-C₆ alkyl-NR₉C(O)OR₁₁, C₀-C₆ alkyl-R₂₆, C₀-C₆ alkyl-R₂₇, or C₀-C₆ alkyl-R₂₈, wherein the alkyl portions of each of the above are optionally substituted with 1, 2, 3, 4, 5 or 6 groups that are independently C₁-C₆ alkyl, hydroxy-C₁-C₆ alkyl, C₁-C₆ alkoxy, C₀-C₆ alkyl-R₂₆, halo, OH, oxo, CF₃, —OCF₃, NO₂, NH₂, mono- or di-(C₁-C₆ alkyl)amino, or CN;
R₂ and R₃ are independently OH, H, NH₂, oxo, mono- or di-(C₁-C₆ alkyl)amino, halo, C₁-C₆ alkyl, C₁-C₆ alkoxy, —C(O)NR₉R₁₀, C₀-C₆ alkyl-C(O)OR₁₁, C₀-C₆ alkyl-R₂₆, C₀-C₆ alkyl-R₂₇, or C₀-C₆ alkyl-R₂₈, wherein the alkyl groups are optionally substituted with 1, 2, 3, 4, 5 or 6 groups that are independently C₁-C₆ alkyl, OH, C₁-C₆ alkoxy, halo, CF₃, —OCF₃, NO₂, NH₂, mono- or di-(C₁-C₆ alkyl)amino, or CN;
R₄, R₅, R₇ and R₈ are independently H, OH, NH₂, mono- or di-(C₁-C₆ alkyl)amino, halo, C₁-C₆ alkoxy, or C₁-C₆ alkyl, wherein the alkoxy and alkyl groups are optionally substituted with 1, 2, 3 or 4 that are independently halo, C₁-C₆ alkyl, C₁-C₆ alkoxy, OH, oxo, CF₃, —OCF₃, NO₂, NH₂, mono- or di-(C₁-C₆ alkyl)amino, or CN;
R₆ is chloro, fluoro, iodo, CF₃, —OCF₃, NO₂, or CN;
R₉ and R₁₀ are independently H, C₁-C₆ alkyl, C₃-C₆ cycloalkyl, or C₀-C₆ alkyl-R₂₆; or
R₉ and R₁₀ together with the nitrogen to which they are attached form pyrrolidinyl or piperidinyl;
R₁₁ is H, C₁-C₆ alkyl, C₁-C₆ cycloalkyl or C₀-C₆ alkyl-R₂₆;
R₁₂ and R₁₃ are independently OH, H, CN, NH₂, C₀-C₆ alkyl-R₂₆, C₀-C₆ alkyl-R₂₇, C₀-C₆ alkyl-R₂₈, —C(O)NR₉R₁₀, mono- or di-(C₁-C₆ alkyl)amino, halo, C₀-C₆ alkyl-C(O)OR₁₁, C₁-C₆ alkyl, or C₁-C₆ alkoxy;
R₁₄ is H, C₁-C₆ alkyl, or oxo;
R₁₅ is C₁-C₆ alkyl, C₁-C₆ alkoxy, hydroxy-C₁-C₆ alkyl, C₁-C₆ alkyl-O-(hydroxy-C₁-C₆ alkyl), —C(O)—N(R₉)₂, —C(O)R₂₇, C₀-C₆ alkyl-C(O)R₂₈, —C(O)OR₁₁, C₀-C₆ alkyl-R₂₆, or C₀-C₆ alkyl-R₂₇, wherein the alkyl groups are optionally substituted with 1, 2, 3, 4, 5 or 6 groups that are independently C₁-C₆ alkyl, C₁-C₆ alkoxy, halo, OH, CF₃, —OCF₃, NO₂, NH₂, mono- or di-(C₁-C₆ alkyl)amino, or CN;
R₁₆ and R₁₇ are independently OH, H, CN, NH₂, C₀-C₆ alkyl-R₂₆, C₀-C₆ alkyl-R₂₇, C₀-C₆ alkyl-R₂₈, —C(O)NR₉R₁₀, mono- or di-(C₁-C₆ alkyl)amino, halo, C₀-C₆ alkyl-C(O)OR₁₁, C₁-C₆ alkyl, or C₁-C₆ alkoxy;
R₁₈ is C₁-C₆ alkyl or oxo;
R₁₉ and R₂₀ are independently OH, H, CN, NH₂, mono- or di-(C₁-C₆ alkyl)amino, halo, C₁-C₆ alkyl, C₁-C₆ alkoxy, C₀-C₆ alkyl-C(O)OR₁₁, —C(O)NR₉R₁₀, C₀-C₆ alkyl-R₂₆, C₀-C₆ alkyl-R₂₇, or C₀-C₆ alkyl-R₂₈, wherein the alkyl groups are optionally substituted with 1, 2, 3 or 4 groups that are independently C₁-C₆ alkyl, OH, C₁-C₆ alkoxy, halo, CF₃, —OCF₃, NO₂, NH₂, mono- or di-(C₁-C₆ alkyl)amino, or CN;
R₂₁ and R₂₂ are independently H, C₁-C₆ alkyl, C₁-C₆ alkoxy, C₁-C₆ alkyl-C₁-C₆ alkoxy, —C(O)OR₁₁, —C(O)NR₉R₁₀, hydroxy C₁-C₆ alkyl, C₀-C₆ alkyl-R₂₈, C₀-C₆ alkyl-R₂₇, C₀-C₆ alkyl-NR₉R₂₆, or —C(O)—O—C₀-C₆ alkyl-R₂₆;
R₂₃ is OH, CN, oxo, NH₂, mono- or di-(C₁-C₆ alkyl)amino, halo, C₁-C₆ alkyl, C₁-C₆ alkoxy, C₁-C₆ alkyl-NR₉R₂₆, C₁-C₆ alkyl-O—C₁-C₆ alkyl, —C(O)R₁₁, —C(O)R₂₇, —C(O)R₂₈, —C(O)NR₉R₁₀, —C(O)OR₁₁, C₀-C₆ alkyl-NR₉C(O)OR₁₁, C₀-C₆ alkyl-R₂₆, C₀-C₆ alkyl-R₂₇, or C₀-C₆ alkyl-R₂₈;
R₂₄ is H or C₁-C₆ alkyl;
R₂₅ is C₁-C₆ alkyl, C₀-C₆ alkyl-NR₉R₂₆, —C(O)O—C₀-C₆ alkyl-R₂₆ or C₀-C₆ alkyl-R₂₈, or C₆ alkyl-R₂₆ wherein the alkyl is optionally substituted with C₀-C₆ alkyl-R₂₆ or OH;
R₂₉ at each occurrence is independently OH, H, CN, halo, C₁-C₆ alkyl, C₁-C₆ alkoxy, —C(O)R₁₁, —C(O)NR₉R₁₀, —C(O)OR₁₁, C₀-C₆ alkyl-NR₉C(O)OR₁₁, C₀-C₆ alkyl-R₂₆, C₀-C₆ alkyl-R₂₇, or C₀-C₆ alkyl-R₂₈, wherein the alkyl portions of each of the above are optionally substituted with 1, 2, 3, 4, 5 or 6 groups that are independently C₁-C₆ alkyl, hydroxy-C₁-C₆ alkyl, C₁-C₆ alkoxy, C₀-C₆ alkyl-R₂₆, halo, OH, oxo, CF₃, —OCF₃, NO₂NH₂, CN, mono- or di-(C₁-C₆ alkyl)amino;
R₃₀ is OH, H, oxo, CN, NH₂, mono- or di-(C₁-C₆ alkyl)amino, halo, C₁-C₆ alkyl, C₁-C₆ alkoxy, C₀-C₆ alkyl-C(O)OR₁₁, —C(O)NR₉R₁₀, C₀-C₆ alkyl-R₂₆, C₀-C₆ alkyl-R₂₇, or C₀-C₆ alkyl-R₂₈, wherein the alkyl groups are optionally substituted with 1, 2, 3 or 4 groups that are independently C₁-C₆ alkyl, OH, C₁-C₆ alkoxy, halo, CF₃, —OCF₃, NO₂, NH₂, mono- or di-(C₁-C₆ alkyl)amino, or CN;
R₂₆ is phenyl which is optionally substituted with 1, 2, 3, 4, or 5 groups that are independently C₁-C₆ alkyl, C₁-C₆ alkoxy, halo, OH, CF₃, —OCF₃, NO₂, NH₂, mono- or di- (C₁-C₆ alkyl)amino, or CN;
R₂₇ is pyridinyl, benzodioxolyl, quinolinyl, pyrimidinyl, furanyl, 1,3-dihydro-2-oxo-benzoimidazol-1-yl, or benzoimidazolyl, each of which is optionally substituted with 1, 2, 3, 4, 5 or 6 groups that are independently C₁-C₆ alkyl, C₁-C₆ alkoxy, halo, OH, CF₃, —OCF₃, NO₂, NH₂, —C(O)N(R₉)₂, —NR₉C(O)N(R₉)₂, —NR₉C(O)OR₉, mono- or di-(C₁-C₆ alkyl)amino, or CN; and
R₂₈ is pyrrolidinyl or piperidinyl, each of which is optionally substituted with 1, 2, 3, 4 or 5 groups that are independently C₁-C₆ alkyl, C₁-C₆ alkoxy, hydroxy-C₁-C₂ alkyl, halo, OH, CF₃, —OCF₃, NO₂, NH₂, mono- or di-(C₁-C₆ alkyl)amino, or CN.

2. The compounds according to claim 1 of the formula 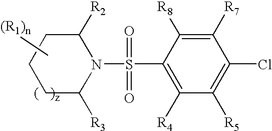
or pharmaceutically acceptable salts thereof, wherein
z is 0, 1, 2, or 3;
n is 0, 1 or 2;
R₁ at each occurrence is independently OH, H, CN, oxo, halo, C₁-C₆ alkyl, C₀-C₄ alkyl-R₂₈, C₀-C₄ alkyl-R₂₆, R₂₇, —C(O)R₁₁, —C(O)NR₉R₁₀, —C(O)OR₁₁, or C₀-C₄ alkyl-NR₉C(O)OR₁₁, wherein each of the alkyl groups is optionally substituted with one or two groups that are independently OH or phenyl;
R₂ and R₃ are independently H, —C(O)NR₉R₁₀, —C(O)OR₁₁, C₀-C₆ alkyl-R₂₆, C₀-C₆ alkyl-R₂₇, or C₀-C₆ alkyl-R₂₈, or C₁-C₆ alkyl, wherein the alkyl group is optionally substituted with OH;
R₄, R₅, R₇ and R₉ are independently H or fluoro;
R₉ and R₁₀ are independently H or C₁-C₆ alkyl;
R₁₁ is H, or C₁-C₆ alkyl;
R₂₆ is phenyl which is optionally substituted with 1, 2 or 3 groups that are independently C₁-C₆ alkyl, C₁-C₆ alkoxy, halo, OH, CF₃, —OCF₃, NO₂, NH₂, mono- or di-(C₁-C₆ alkyl)amino, or CN;
R₂₇ is pyridinyl, 1,3-dihydro-2-oxo-benzoimidazol-1-yl, benzodioxolyl, quinolinyl, pyrimidinyl, furanyl, or benzoimidazolyl, each of which is optionally substituted with 1, 2 or 3 groups that are independently C₁-C₆ alkyl, C₁-C₆ alkoxy, halo, OH, CF₃, —OCF₃, NO₂, NH₂, —C(O)N(R₉)₂, —NR₉C(O)N(R₉)₂, —NR₉C(O)OR₉, mono- or di-(C₁-C₆ alkyl)amino, or CN; and
R₂₈ is pyrrolidinyl or piperidinyl, each of which is optionally substituted with 1, 2 or 3 groups that are independently hydroxy-C₁-C₄ alkyl, C₁-C₆ alkyl, C₁-C₆ alkoxy, halo, OH, CF₃, —OCF₃, NO₂, NH₂, mono- or di-(C₁-C₆ alkyl)amino, or CN.

3. The compounds according to claim 2 wherein at least one of R₁, R₅, R₄, R₇, and R₈ is H, and R₂ and R₃ are independently H, R₂₇, or C₁-C₆ alkyl optionally substituted with OH.

4. The compounds according to claim 3 wherein R₁, R₅, R₄, R₇, and R₈ are H.

5. The compounds according to claim 4 wherein R₂ is H and R₃ is H, R₂₇, or C₁-C₆ alkyl optionally substituted with OH.

6. The compounds according to claim 5 wherein R₃ is C₁-C₄-alkyl.

7. The compounds of claim 5 wherein R₃ is pyridinyl, quinolinyl, pyrimidinyl, or furanyl.

8. The compounds according to claim 3 wherein R₂ and R₃ are independently C₁-C₄ alkyl.

9. The compounds according to claim 3 wherein R₂ and R₃ are H.

10. The compounds according to claim 2 wherein at least one of R₄, R₅, R₈, R₇, R₂, and R₃ is H, n is 1, and R₁ is OH, halo, or C₁-C₆ alkyl optionally substituted with OH.

11. The compounds according to claim 8 wherein R₄, R₅, R₈, R₇, R₂, and R₃ are H.

12. The compounds according claim 11 wherein n is 1 or 2, and each R₁ is independently methyl or propyl.

13. The compounds according to claim 3, wherein R₂, R₃ are independently H, or C₁-C₆ alkyl; and z is 2.

14. A compounds according to claim 1 of the formula 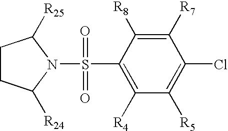
or pharmaceutically acceptable salts thereof, wherein
R₄, R₅, R₇ and R₈ are independently H or fluoro;
R₂₄ is H or C₁-C₄ alkyl; and
R₂₅ is C₁-C₄ alkyl, C₀-C₄ alkyl-NH-phenyl, —C(O)O—C₀-C₄ alkyl-phenyl, C₀-C₄ alkyl-pyrrolidinyl, or C₀-C₄ alkyl-phenyl wherein the alkyl portion is optionally substituted with phenyl and OH.

15. The compounds according to claim 14 wherein R₄, R₅, R₈, and R₇ are H.

16. The compounds according to claim 15 wherein R₂₄ and R₂₅ are C₁-C₄ alkyl.

17. The compounds according to claim 1 of the formulas 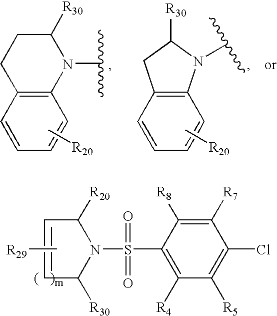
or pharmaceutically acceptable salts thereof, wherein
m is 1 or 2;
R₂₉ is H, C₁-C₆ alkyl, or C₁-C₆ alkoxy;
R₄, R₅, R₇ and R₈ are independently H or fluoro; and
R₃₀ and R₂₀ are independently H, C₁-C₄ alkyl, or C₁-C₆ alkoxy; or
R₃₀ is C(O)NR₉R₁₀, where R₉ and R₁₀ are independently H, C₁-C₄-alkyl, or C₀-C₆ alkyl-R₂₆.

18. The compounds according to claim 17 wherein at least one of R₄, R₅, R₈, R₇ and R₂₉ is H, m is 1, and R₃₀ and R₂₀ are independently H or C₁-C₄ alkyl.

19. The compounds according to claim 18 wherein R₄, R₅, R₈, R₇ and R₂₉ are H.

20. The compounds according to claim 19 wherein R₂₀ is C₁-C₄ alkyl and R₃₀ is C₁-C₄ alkyl or -CH₂-R₂₆.

21. A pharmaceutical composition comprising a compound or salt of claim 1 and at least one pharmaceutically acceptable carrier, solvent, adjuvant or excipient, or a combination thereof.

22. A method of treating a patient who has, or in preventing or delaying a patient from getting, a disease or condition selected from the group consisting of Alzheimer's disease (AD), mild cognitive impairment (MCI), Down's syndrome, Hereditary Cerebral Hemorrhage with Amyloidosis of the Dutch-Type, cerebral amyloid angiopathy and its potential consequences, i.e. single and recurrent lobar hemorrhages, other degenerative dermentias, including dementias of mixed vascular and degenerative origin, dementia associated with Parkinson's disease, dementia associated with progressive supranuclear palsy, dementia associated with cortical basal degeneration, age related macular degeneration, or diffuse Lewy body type of Alzheimer's disease and who is in need of such treatment which comprises administration of a therapeutically effective amount of a compound or salt of claim 1.

23. A compounds according to claim 1 that is
2-(4-Chloro-benzenesulfonyl)-6-methoxy-2,3,4,9-tetrahydro-1H-b-carboline;
8-(4-Chloro-benzenesulfonyl)-1,4-dioxa-8-aza-spiro[4.5]decane;
1-(4-Chloro-benzenesulfonyl)-1,3,4,6,7,8-hexahydro-2H-pyrimido[1,2-a]pyrimidine;
(3R)-N-(tert-butyl)-2-[(4-chlorophenyl)sulfonyl]decahydroisoquinoline-3-carboxamide;
1′-[(4-chlorophenyl)sulfonyl]spiro[indene-1,4′-piperidine];
(2S)-1-[(4-chlorophenyl)sulfonyl]octahydro-1H-indole-2-carboxylic acid;
8-(4-Chloro-benzenesulfonyl)-1-phenyl-1,3,8-triaza-spiro[4.5]decan-4-one;
[8-(4-Chloro-benzenesulfonyl)-7,7,9,9-tetramethyl-1,4-dioxa-8-aza-spiro[4.5]dec-2-yl]-methanol;
1-(4-Chloro-benzenesulfonyl)-decahydro-quinoline;
(1s,5s)-3-[(4-chlorophenyl)sulfonyl]-3-azabicyclo[3.2.2]nonane;
2-(4-Chloro-benzenesulfonyl)-1-methyl-2,3,4,9-tetrahydro-1H-b-carboline-3-carboxylic acid;
1-(4-Chloro-benzenesulfonyl)-2,3-dihydro-1H-indole-2-carboxylic acid benzylamide;
1-(4-Chloro-benzenesulfonyl)-6-fluoro-2-methyl-1,2,3,4-tetrahydro-quinoline;
1-(4-Chloro-benzenesulfonyl)-2,3-dihydro-1H-indole-2-carboxylic acid butylamide;
5-(4-Chloro-benzenesulfonyl)-10,11-dihydro-5H-dibenzo[b,f]azepine;
5-(4-Chloro-benzenesulfonyl)-5H-dibenzo[b,f]azepine;
4-(4-Chloro-benzenesulfonyl)-3-methyl-3,4-dihydro-2H-benzo[1,4]oxazine;
4-(4-Chloro-benzenesulfonyl)-3-methyl-3,4-dihydro-1H-quinoxalin-2-one;
1-(4-Chloro-benzenesulfonyl)-2,3-dihydro-1H-indole-2-carboxylic acid phenylamide;
4-(4-Chloro-benzenesulfonyl)-3-phenyl-3,4-dihydro-2H-benzo[1,4]oxazine;
1-(4-Chloro-benzenesulfonyl)-1,2,3,4-tetrahydro-quinoline;
4-(4-Chloro-benzenesulfonyl)-3,4-dihydro-1H-quinoxalin-2-one;
2-(4-Chloro-benzenesulfonyl)-1,2,3,4-tetrahydro-isoquinoline;
1-(4-Chloro-benzenesulfonyl)-2,3-dihydro-1H-indole-2-carboxylic acid;
1-(4-Chloro-benzenesulfonyl)-5-methoxy-1H-indole-2-carboxylic acid;
1-(4-Chloro-benzenesulfonyl)-2,3-dihydro-1H-indole-2-carboxylic acid amide;
1-(4-Chloro-benzenesulfonyl)-2,3-dihydro-1H-indole-2-carboxylic acid methylamide;
1-(4-Chloro-benzenesulfonyl)-2,3-dihydro-1H-indole-2-carboxylic acid dimethylamide;
[1-(4-Chloro-benzenesulfonyl)-2,3-dihydro-1H-indol-2-yl]-pyrrolidin-1-yl-methanone;
1-(4-Chloro-benzenesulfonyl)-2,3-dihydro-1H-indole-2-carboxylic acid benzyl-methyl-amide;
10-(4-Chloro-benzenesulfonyl)-10H-phenothiazine
1-(4-Chloro-benzenesulfonyl)-6-ethoxy-2,2,4-trimethyl-1,2-dihydro-quinoline;
1-(4-Chloro-benzenesulfonyl)-1H-naphtho[1,2-d]imidazol-7-ol;
1-(4-Chloro-benzenesulfonyl)-1,2,3,4-tetrahydro-quinolin-8-ylamine;
1-(4-Chloro-benzenesulfonyl)-5-nitro-2,3-dihydro-1H-indole;
2-(4-Chloro-benzenesulfonyl)-6,7-dimethoxy-1,2,3,4-tetrahydro-isoquinoline;
1-(4-Chloro-benzenesulfonyl)-6-nitro-2,3-dihydro-1H-indole;
[2-(4-Chloro-benzenesulfonyl)-1,2,3,4-tetrahydro-isoquinolin-1-yl]-acetic acid;
1-(4-Chloro-benzenesulfonyl)-2-methylsulfanyl-1H-benzoimidazole;
1-(4-Chloro-benzenesulfonyl)-1H-indazole;
10-(4-Chloro-benzenesulfonyl)-1,4,7-trioxa-10-aza-cyclododecane;
1-(4-Chloro-benzenesulfonyl)-[1,4]diazepane;
1-(4-Chloro-benzenesulfonyl)-azepane;
1-(4-Chloro-benzenesulfonyl)-piperidine;
(2R,6S)-1-[(4-chlorophenyl)sulfonyl]-2,6-dimethylpiperidine;
1-(4-Chloro-benzenesulfonyl)-2-ethyl-piperidine;
1-(4-Chloro-benzenesulfonyl)-2,6-dimethyl-piperidine;
1-(4-Chloro-benzenesulfonyl)-2-methyl-piperidine;
[1-(4-Chloro-benzenesulfonyl)-piperidin-2-yl]-methanol;
1-(4-Chloro-benzenesulfonyl)-1,2,3,4,5,6-hexahydro-[2,3′]bipyridinyl;
1-(4-Chloro-benzenesulfonyl)-3,5-dimethyl-piperidine;
1-(4-Chloro-benzenesulfonyl)-4-methyl-piperidine;
2-[1-(4-Chloro-benzenesulfonyl)-piperidin-4-yl]-ethanol;
1-(4-Chloro-benzenesulfonyl)-3-methyl-piperidine;
1-(4-Chloro-benzenesulfonyl)-piperidin-4-ol;
4-Bromo-1-(4-chloro-benzenesulfonyl)-piperidine;
1-(4-Chloro-benzenesulfonyl)-piperidin-3-ol;
[1-(4-Chloro-benzenesulfonyl)-piperidin-3-yl]-methanol;
[1-(4-Chloro-benzenesulfonyl)-piperidin-4-yl]-methanol;
1-(4-Chloro-benzenesulfonyl)-4-propyl-piperidine;
1-(4-Chloro-benzenesulfonyl)-piperidine-3-carboxylic acid;
1-(4-Chloro-benzenesulfonyl)-piperidine-4-carboxylic acid;
1-(4-Chloro-benzenesulfonyl)-piperidine-3-carboxylic acid diethylamide;
1-(4-Chloro-benzenesulfonyl)-piperidine-4-carboxylic acid ethyl ester;
1-(4-Chloro-benzenesulfonyl)-piperidine-3-carboxylic acid ethyl ester;
{2-[1-(4-Chloro-benzenesulfonyl)-piperidin-4-yl]-ethyl}-carbamic acid tert-butyl ester;
1-(4-Chloro-benzenesulfonyl)-4-phenyl-piperidine;
4-Benzyl-1-(4-chloro-benzenesulfonyl)-piperidine;
1′-(4-Chloro-benzenesulfonyl)-[1,4′]bipiperidinyl;
2-(4-{3-[1-(4-Chloro-benzenesulfonyl)-piperidin-4-yl]-propyl}-piperidin-1-yl)-ethanol;
[1-(4-Chloro-benzenesulfonyl)-piperidin-4-yl]-diphenyl-methanol;
1-[1-(4-Chloro-benzenesulfonyl)-piperidin-4-yl]-1,3-dihydro-benzoimidazol-2-one;
1-(4-Chloro-benzenesulfonyl)-4-oxo-piperidine-3-carboxylic acid ethyl ester;
1-(4-Chloro-benzenesulfonyl)-3-methyl-3-phenyl-piperidine;
1-(4-Chloro-benzenesulfonyl)-4-(4-chloro-phenyl)-piperidin-4-ol;
1-(4-Chloro-benzenesulfonyl)-4-phenyl-piperidine-4-carbonitrile;
1-(4-Chloro-benzenesulfonyl)-4-phenyl-piperidin-4-ol;
1-[1-(4-Chloro-benzenesulfonyl)-4-phenyl-piperidin-4-yl]-ethanone;
[1-(4-Chloro-benzenesulfonyl)-3-oxo-piperazin-2-yl]-acetic acid ethyl ester;
(3S)-1-[(4-chlorophenyl)sulfonyl]-3-methylpiperazine;
(3R)-1-[(4-chlorophenyl)sulfonyl]-3-methylpiperazine;
1-(4-Chloro-benzenesulfonyl)-4-ethyl-piperazine
2-[4-(4-Chloro-benzenesulfonyl)-piperazin-1-yl]-ethanol;
2-{2-[4-(4-Chloro-benzenesulfonyl)-piperazin-1-yl]-ethoxy}-ethanol;
4-(4-Chloro-benzenesulfonyl)-piperazine-1-carboxylic acid ethyl ester;
4-(4-Chloro-benzenesulfonyl)-piperazine-1-carboxylic acid tert-butyl ester;
2-[4-(4-Chloro-benzenesulfonyl)-piperazin-1-yl]-1-pyrrolidin-1-yl-ethanone;
[4-(4-Chloro-benzenesulfonyl)-piperazin-1-yl]-furan-2-yl-methanone;
4-(4-Chloro-benzenesulfonyl)-piperazine-1-carboxylic acid benzyl ester;
1-Benzyl-4-(4-chloro-benzenesulfonyl)-piperazine;
4-(4-Chloro-benzenesulfonyl)-2-methyl-1-phenyl-piperazine;
1-(4-Chloro-benzenesulfonyl)-4-(4-chloro-benzyl)-piperazine;
1-(4-Chloro-benzenesulfonyl)-4-o-tolyl-piperazine;
4-(4-Chloro-benzenesulfonyl)-2-methyl-1-p-tolyl-piperazine;
4-(4-Chloro-benzenesulfonyl)-1-(4-methoxy-phenyl)-2-methyl-piperazine;
1-(4-Chloro-benzenesulfonyl)-4-(2-methoxy-phenyl)-piperazine;
1-(4-Chloro-benzenesulfonyl)-4-(4-fluoro-phenyl)-piperazine;
1-(4-Chloro-benzenesulfonyl)-4-(3-chloro-phenyl)-piperazine;
1-(4-Chloro-benzenesulfonyl)-4-(3,4-dichloro-phenyl)-piperazine;
1-(4-Chloro-benzenesulfonyl)-4-(3,5-dichloro-phenyl)-piperazine;
1-(4-Chloro-benzenesulfonyl)-4-phenethyl-piperazine;
1-Benzhydryl-4-(4-chloro-benzenesulfonyl)-piperazine;
1-(4-Chloro-benzenesulfonyl)-4-( 1-phenyl-ethyl)-piperazine;
1-(4-Chloro-benzenesulfonyl)-4-(3-trifluoromethyl-phenyl)-piperazine;
1-(4-Chloro-benzenesulfonyl)-4-(4-chloro-phenyl)-piperazine;
2-[4-(4-Chloro-benzenesulfonyl)-piperazin-1-yl]-benzonitrile;
1-(4-Chloro-benzenesulfonyl)-4-(2,3-dimethyl-phenyl)-piperazine;
1-(4-Chloro-benzenesulfonyl)-4-p-tolyl-piperazine;
4-(4-Chloro-benzenesulfonyl)-2-methyl-1-m-tolyl-piperazine;
1-Benzo[1,3]dioxol-5-ylmethyl-4-(4-chloro-benzenesulfonyl)-piperazine;
4-[4-(4-Chloro-benzenesulfonyl)-piperazin-1-yl]-2-trifluoromethyl-quinoline;
2-[4-(4-Chloro-benzenesulfonyl)-piperazin-1-yl]-pyrimidine;
1-(4-Chloro-benzenesulfonyl)-4-pyridin-4-yl-piperazine;
1-(4-Chloro-benzenesulfonyl)-4-pyridin-2-yl-piperazine;
1-(4-Chloro-benzenesulfonyl)-4-(5-trifluoromethyl-pyridin-2-yl)-piperazine;
4-(4-Chloro-benzenesulfonyl)-2,6-dimethyl-morpholine;
4-(4-Chloro-benzenesulfonyl)-morpholine;
4-(4-Chloro-benzenesulfonyl)-thiomorpholine;
1-(4-Chloro-benzenesulfonyl)-pyrrolidin-3-ol;
(3S)-1-[(4-chlorophenyl)sulfonyl]pyrrolidin-3-ol;
tert-butyl{(3S)-1-[(4-chlorophenyl)sulfonyl]pyrrolidin-3-yl}carbamate;
tert-butyl{(3R)-1-[(4-chlorophenyl)sulfonyl]pyrrolidin-3-yl}carbamate;
[1-(4-Chloro-benzenesulfonyl)-pyrrolidin-3-yl]-carbamic acid tert-butyl ester;
1-(4-Chloro-benzenesulfonyl)-2,5-dimethyl-pyrrolidine;
(2S)-1-[(4-chlorophenyl)sulfonyl]-2-(pyrrolidin-1-ylmethyl)pyrrolidine;
benzyl 1-[(4-chlorophenyl)sulfonyl]-D-prolinate;
benzyl 1-[(4-chlorophenyl)sulfonyl]-L-prolinate;
N-({(2R)-1-[(4-chlorophenyl)sulfonyl]pyrrolidin-2-yl}methyl)aniline;
{(2S)-1-[(4-chlorophenyl)sulfonyl]pyrrolidin-2-yl}(diphenyl)methanol;
(2R,5R)-1-[(4-chlorophenyl)sulfonyl]-2,5-dimethylpyrrolidine;
1-(4-Chloro-benzenesulfonyl)-5,5-diphenyl-imidazolidine-2,4-dione;
1-(4-Chloro-benzenesulfonyl)-5-phenyl-5-p-toly-imidazolidine-2,4-dione;
1-(4-Chloro-benzenesulfonyl)-5-(3-hydroxy-phenyl)-5-phenyl-imidazolidine-2,4-dione;
3-(4-Chloro-benzenesulfonyl)-4,4-dimethyl-oxazolidine;
3-(4-Chloro-benzenesulfonyl)-thiazolidine
(4S)-3-[(4-chlorophenyl)sulfonyl]-2-(3,4-dimethoxyphenyl)-N-phenyl-1,3-thiazolidine-4-carboxamide;
(4R)-N-(sec-butyl)-3-[(4-chlorophenyl)sulfonyl]-2-(3,4-dimethoxyphenyl)-1,3-thiazolidine-4-carboxamide;
(4S)-N-benzyl-3-[(4-chlorophenyl)sulfonyl]-2-(3,4-dimethoxyphenyl)-1,3-thiazolidine-4-carboxamide;
(4S)-N-butyl-3-[(4-chlorophenyl)sulfonyl]-2-(3,4-dimethoxyphenyl)-1,3-thiazolidine-4-carboxamide;
(4R)-N-butyl-3-[(4-chlorophenyl)sulfonyl]-2-(3,4-dimethoxyphenyl)-1,3-thiazolidine-4-carboxamide;
(4R)-3-[(4-chlorophenyl)sulfonyl]-N-cyclohexyl-2-(3,4-dimethoxyphenyl)-1,3-thiazolidine-4-carboxamide;
(4S)-3-[(4-chlorophenyl)sulfonyl]-2-(3,4-dimethoxyphenyl)-N-phenyl-1,3-thiazolidine-4-carboxamide;
(4S)-N-benzyl-3-[(4-chlorophenyl)sulfonyl]-2-(3,4-dimethoxyphenyl)-1 ,3-thiazolidine-4-carboxamide;
(4S)-3-[(4-chlorophenyl)sulfonyl]-N-cyclohexyl-2-(3,4-dimethoxyphenyl)-1,3-thiazolidine-4-carboxamide;
2-(4-Chloro-benzenesulfonyl)-4-iodo-2H-pyrazole-3-carboxylic acid;
4-Bromo-1-(4-chloro-benzenesulfonyl)-3-methyl-1H-pyrazole;
4-Chloro-2-[1-(4-chloro-benzenesulfonyl)-1H-pyrazol-3-yl]-phenol;
1-(4-Chloro-benzenesulfonyl)-1,2,3,6-tetrahydro-pyridine;
1-(4-Chloro-benzenesulfonyl)-2,5-dimethyl-2,5-dihydro-1H-pyrrole;
1-(4-Chloro-benzenesulfonyl)-2,5-dihydro-1H-pyrrole;
1-(4-Chloro-benzenesulfonyl)-azetidine;
1-(4-Chloro-benzenesulfonyl)-2-phenyl-aziridine; or
pharmaceutically acceptable salts thereof.
